(19) United States
(12) Patent Application Publication
(10) Pub. No.: US 2010/0260849 A1
(43) Pub. Date: Oct. 14, 2010

(54) REMINERALIZING COMPOSITIONS AND METHODS

(76) Inventors: Richard P. Rusin, Woodbury, MN (US); Kevin M. Cummings, Little Canada, MN (US); Sumita B. Mitra, West St. Paul, MN (US)

Correspondence Address: 3M INNOVATIVE PROPERTIES COMPANY, PO BOX 33427, ST. PAUL, MN, 55133-3427, US

(21) Appl. No.: 12/747,546

(22) PCT Filed: Dec. 11, 2008
(86) PCT No.: PCT/US08/86327

§ 371 Date: Jun. 11, 2010

Related U.S. Application Data
(60) Provisional application No. 61/013,464, filed on Dec. 13, 2007.

(51) Int. Cl. A61K 8/04; A61K 8/24; A61Q 90/00; A61Q 11/00

(52) U.S. Cl. 424/486; 424/601

(57) ABSTRACT

The present application provides compositions comprising a divalent metal cation source or divalent metal cations dissolved in a substantially anhydrous liquid, and methods of making and using the compositions. Such compositions can be useful for remineralizing dental structures and/or providing other useful effects including an anticaries effect.

### CROSS REFERENCE TO RELATED APPLICATIONS

The present invention claims priority to U.S. Provisional Application Ser. No. 61/013,464, filed Dec. 13, 2007, which is incorporated herein by reference.

### BACKGROUND

Demineralization of dental structures is well known to lead to caries, decayed dentin, cementum, and/or enamel, conditions that typically require treatment with a dental restorative, for example. Although such conditions can usually be adequately treated using dental restoratives, restored dental structures oftentimes can be susceptible to further decay around the margins of the restoration.

The release of ions (e.g., calcium, and preferably calcium and phosphate ions) into the oral environment is known to enhance the natural remineralizing capability of dental structures. It is believed that enhanced remineralization may be a useful supplement to, or even an alternative to, traditional dental restorative methods. However, known compositions that release calcium and phosphorus into the oral environment (e.g., calcium phosphate containing compositions) may lack desirable properties.

Thus, there is a continuing need for new compositions capable of releasing ions (e.g., calcium and other ions) into the oral environment.

### SUMMARY OF THE INVENTION

The present invention provides compositions comprising a divalent metal cation source or divalent metal cations dissolved in a substantially anhydrous liquid. In some embodiments, these compositions can be advantageous, because they are not susceptible to drying. In some embodiments, components that would react with each other in water can be co-solubilized in a stable solution in the substantially anhydrous liquid. In some embodiments, because the ion sources are solubilized, the ions are immediately available as compared with ion sources in solid forms, such as particles, which may first undergo dissolution or elution in situ. In some embodiments, single-component systems are easier for the user than multi-component systems that may involve mixing. Such compositions can be used for remineralizing dental structures and/or providing other useful effects, for example, an anticaries effect, an antibacterial effect, increased x-ray opacity, or imparting fluorescence similar to the dental structure for improved esthetics or fluorescence distinct from the dental structure to aid detection.

In one embodiment, the present invention provides a remineralizing composition comprising:

a calcium source and a phosphorous source, each of which is dissolved in a substantially anhydrous liquid; and

a matrix forming component selected from the group consisting of a polymerizable resin, a film former and a combination thereof;

wherein the matrix forming component is dissolved in the substantially anhydrous liquid, and wherein the matrix forming component optionally comprises a portion of the substantially anhydrous liquid; or

wherein the matrix forming component is the substantially anhydrous liquid.

In another embodiment, there is provided a remineralizing composition comprising a calcium source, a phosphorous source, and at least one cation selected from the group consisting of cations of Zn, Sn, and Ag, wherein the calcium source, the phosphorous source, and the at least one cation are dissolved in a substantially anhydrous liquid.

In another embodiment, there is provided a remineralizing composition comprising at least one divalent metal cation and a phosphate anion, both of which are dissolved in a substantially anhydrous liquid;

wherein the phosphate anion is selected from the group consisting of (P₂O₇)⁻⁴, (H₂PO₂)⁻¹, and an anion represented by the formula: H—[CH(—OR)—]ₓH, wherein x is an integer from 2 to 4; and each R is independently H or —P(O)(O⁻)₂, and wherein at least one R is —P(O)(O⁻)₂.

In another embodiment, there is provided a composition comprising a divalent metal cation source and an organic anticaries agent, both of which are dissolved in a substantially anhydrous liquid, wherein the divalent metal cation is selected from the group consisting of cations of Ca, Zn, Sr, Ba, and Mg.

In another embodiment, there is provided a dental bleach composition comprising a calcium source, a phosphorous source, and a bleaching agent, all of which are dissolved in a substantially anhydrous liquid.

In another embodiment, there is provided a two-part remineralizing composition comprising:

a first part comprising a calcium source and a phosphorous source, both of which are dissolved in a substantially anhydrous liquid; and

a second part comprising an orally acceptable liquid or paste.

In another embodiment, there is provided a remineralizing composition comprising:

at least one particulate source of calcium and phosphorous combined with a substantially anhydrous liquid, wherein the at least one particulate source is selected from the group consisting of a glass, a glass-ceramic, active treated particles, nanoparticles, nanoclusters, amorphous calcium phosphate, and a combination thereof, and wherein the at least one particulate source includes calcium, phosphorous, or calcium and phosphorous, which can be released; and

at least one of:
a divalent metal salt dissolved in the substantially anhydrous liquid, and
a phosphate source dissolved in the substantially anhydrous liquid.

In one aspect, the present invention also provides a method of preparing a remineralizing composition comprising:

dissolving a phosphate anion in a substantially anhydrous liquid to provide a first solution; wherein the phosphate anion is selected from the group consisting of (P₂O₇)⁻⁴, (H₂PO₂)⁻¹, and an anion represented by the formula: H—[CH(—OR)—]ₓH, wherein x is an integer from 2 to 4; and each R is independently H or —P(O)(O⁻)₂, and wherein at least one R is —P(O)(O⁻)₂;

dissolving at least one divalent metal cation separately from the phosphate anion in the substantially anhydrous liquid to form a second solution; and combining the first and second solutions.

In another aspect, methods of using the above compositions for treating a tooth structure, for remineralizing a tooth structure, for reducing the sensitivity of a tooth structure, for protecting a tooth structure, for delivering a plurality of ions to an oral environment, and for preparing a dental article are also provided.

In another aspect, there is provided a kit comprising any one of the above compositions; and an applicator.

In some embodiments, compositions disclosed herein are preferably dental compositions which lead to enhanced remineralization of dental structures, which can offer potential benefits including, for example, the ability to remineralize enamel and/or dentin lesions; to occlude exposed dentin and/or cementum tubules which cause sensitivity; to recondition abraded and/or etched enamel surfaces; to reseal microleakage regions at interfaces; and/or to increase resistance of contacted and nearby tooth structures to acid attack. In some embodiments, dental compositions as disclosed herein have antimicrobial behavior, which can act against bacteria that cause decay.

### DEFINITIONS

As used herein, a “substantially anhydrous liquid” refers to a liquid to which water has not been added as a component. However, there may be adventitious water, such as water of hydration or water present as a coordination complex, associated with one or more materials dissolved in the substantially anhydrous liquid. Water taken up by hygroscopic materials or present as a hydrate may be present in the compositions described herein. Any water that is present in the substantially anhydrous liquid should not be present in amounts such that the water would have a deleterious effect on the long term properties of the composition. For example, the amount of water should be sufficiently low so that the water does not adversely affect stability (e.g., the shelf-life) of a composition comprising the substantially anhydrous liquid having the one or more materials dissolved therein. Adverse effects on stability may include the appearance of lumpiness or graininess in the composition. The substantially anhydrous liquid preferably includes less than 1% by weight, more preferably less than 0.5% by weight, and most preferably less than 0.1% by weight water, based on the total weight of the composition comprising the substantially anhydrous liquid having the one or more materials dissolved therein.

As used herein, “dissolved in a substantially anhydrous liquid” refers to a solution wherein one or more materials dissolved in the substantially anhydrous liquid form a single phase solution, such that the solution appears by visual inspection to be clear. For example, ion sources that are dissolved in the substantially anhydrous liquid form a clear, single-phase solution in which no precipitate, undissolved matter, cloudiness, or separation is visually observed. This observation is made without the presence of insoluble, opacifying, or coloring ingredients such as fillers, abrasives, or pigments, which would interfere with the observation.

As used herein, “dental structures” refers to tooth structures and bone. The term “tooth structures” refers to enamel, dentin, and cementum.

As used herein, “dental material” refers to a material that may be bonded to a dental structure surface and includes, for example, dental restoratives, orthodontic appliances, and/or orthodontic adhesives.

As used herein, “adhesive” or “dental adhesive” refers to a composition used as a pre-treatment on a dental structure (e.g., a tooth) to adhere a “dental material” (e.g., “restorative,” an orthodontic appliance (e.g., bracket), or an “orthodontic adhesive”) to the dental structure. An “orthodontic adhesive” refers to a composition used to adhere an orthodontic appliance to a dental structure (e.g., tooth) surface. Orthodontic adhesives may be highly filled, for example, greater than 20% by weight filler. Generally, the dental structure surface is pre-treated, e.g., by etching, priming, and/or applying an adhesive to enhance the adhesion of the “orthodontic adhesive” to the dental structure surface.

As used herein, “hardening” or “curing” a composition are used interchangeably and refer to polymerization and/or crosslinking reactions including, for example, photopolymerization reactions and chemical polymerization techniques (e.g., ionic reactions or chemical reactions forming radicals effective to polymerize ethylenically unsaturated compounds) involving one or more compounds capable of hardening or curing.

As used herein, “rare earth” (RE) refers to a rare earth element (i.e., an element having an atomic number of 39 or 57-71, inclusive). Rare earth elements include, for example, cerium (Ce), dysprosium (Dy), erbium (Er), europium (Eu), gadolinium (Gd), holmium (Ho), lanthanum (La), lutetium (Lu), neodymium (Nd), praseodymium (Pr), samarium (Sm), terbium (Tb), thulium (e.g., Tm), ytterbium (Yb), yttrium (Y), and combinations thereof.

As used herein, an “amorphous” material is one which does not give rise to a discernible x-ray powder diffraction pattern. An “at least partially crystalline” material is one which gives rise to a discernible x-ray powder diffraction pattern.

As used herein, “groups” of the periodic table refer to and include groups 1-18 as defined in IUPAC Nomenclature of Inorganic Chemistry, Recommendations 1990.

As used herein, “(meth)acryl” is a shorthand term referring to “acryl” and/or “methacryl.” For example, a “(meth)acryloxy” group is a shorthand term referring to either an acryloxy group (i.e., CH₂═CHC(O)O—) and/or a methacryloxy group (i.e., CH₂═C(CH₃)C(O)O—).

As used herein, “ion source” and “ion source compound” refer to a substance that comprises a desired element in the form of or as part of an ion, or in a form which can produce an ion containing the element. Such ions include, for example, calcium ion, metal cation, divalent metal cation, phosphate anion, fluoride ion, various phosphate ions (e.g., hydrogen phosphate, dihydrogen phosphate, glycerophosphate, hexafluorophosphate, etc.), various pyrophosphate ions (e.g., hydrogen pyrophosphate, dihydrogen pyrophosphate, trihydrogen pyrophosphate), and the like. Ion sources and ion source compounds include, for example, calcium sources, phosphorous sources, sources of at least one metal cation, sources of cations of Zn, Sn, and Ag, sources of at least one divalent metal cation, sources of a phosphate anion, fluoride sources, and the like. Table 1 lists some examples of ion sources.

As used herein, “a”, “an”, “the”, “at least one”, and “one or more” are used interchangeably.

The terms “comprises” and variations thereof do not have a limiting meaning where these terms appear in the description and claims.

The above summary of the present invention is not intended to describe each disclosed embodiment or every implementation of the present invention. The description that follows more particularly exemplifies illustrative embodiments. In several places throughout the description, guidance is provided through lists of examples, which examples can be used in various combinations. In each instance, the recited list serves only as a representative group and should not be interpreted as an exclusive list.

### BRIEF DESCRIPTION OF THE DRAWINGS

FIG. 1 is a scanning electron micrograph of exposed dentin treated with a composition of the present invention, which shows partial occlusion of the dentin tubules after one treatment.

FIG. 2 is a scanning electron micrograph of exposed dentin treated with another composition of the present invention, which shows partial occlusion of the dentin tubules after one treatment.

FIG. 3 is a scanning electron micrograph of exposed dentin treated with another composition of the present invention, which shows partial occlusion of the dentin tubules after one treatment.

FIG. 4 is a scanning electron micrograph of untreated exposed dentin, which shows no occlusion of the dentin tubules.

FIG. 5 is a scanning electron micrograph of exposed dentin treated with another composition of the present invention, which shows partial occlusion of the dentin tubules after one treatment.

FIG. 6 is a scanning electron micrograph of exposed dentin treated with another composition of the present invention, which shows partial occlusion of the dentin tubules after one treatment.

FIG. 7 is a scanning electron micrograph of untreated exposed dentin, which shows no occlusion of the dentin tubules.

### DETAILED DESCRIPTION OF ILLUSTRATIVE EMBODIMENTS OF THE INVENTION

The present invention provides compositions comprising a divalent metal cation source, such as a divalent metal salt, or divalent metal cations dissolved in a substantially anhydrous liquid. Such divalent metal cations include, for example, the divalent cations of Ca, Zn, Sn, Sr, Mg, and Ba. In certain embodiments, remineralizing compositions preferably include divalent calcium cations or a source thereof dissolved in a substantially anhydrous liquid. In certain of these embodiments, a phosphorous source dissolved in a substantially anhydrous liquid is included. In certain embodiments, the compositions preferably include or further include divalent metal cations or a source thereof dissolved in the substantially anhydrous liquid. Such divalent metal cations include, for example, the divalent cations of Zn, Sr, Mg, Ba, and combinations thereof, which can enhance remineralization activity, and the divalent cations of Zn, Sn, and a combination thereof, which can provide antibacterial activity.

In one embodiment, the present invention provides a remineralizing composition comprising a calcium source and a phosphorous source, each of which is dissolved in a substantially anhydrous liquid; and

a matrix forming component selected from the group consisting of a polymerizable resin, a film former and a combination thereof;

wherein the matrix forming component is dissolved in the substantially anhydrous liquid, and wherein the matrix forming component optionally comprises a portion of the substantially anhydrous liquid; or

wherein the matrix forming component is the substantially anhydrous liquid.

For certain embodiments, the above remineralizing composition further comprises at least one metal cation selected from the group consisting of cations of Mg, Sr, Ba, Sn, Zn, Zr, La, Al, and Ag. The cations of magnesium, strontium, barium, and zinc may enhance remineralization activity. The cations of tin, zinc, and silver may provide antibacterial benefits. Tin cations may provide an antigingivits benefit. Zinc cations may alleviate halitosis. Cations with atomic number 30 or greater can provide radiopacity. Zirconium and lanthanum cations can provide fluorescence. Strontium chloride can serve as a nerve calming agent. Additional cation sources may also be included. For example, rare earth sources can provide fluorescence; potassium nitrate can serve as a nerve calming agent; and aluminum can complex with a polycarboxylate in an ionomeric setting reaction.

For certain embodiments, including any one of the above embodiments, the composition is a one-part composition. Alternatively, for certain embodiments, the composition is a two-part composition, wherein the calcium source is in one part and the phosphorous source is in the other part.

For certain embodiments, including any one of the above embodiments, the matrix forming component is a polymerizable resin. Such polymerizable resins are described herein below. Alternatively, for certain embodiments, the matrix forming component is a film former. Such film formers are described herein below. Alternatively, for certain embodiments, the matrix forming component is a combination of a polymerizable resin and a film former.

For certain embodiments, including any one of the above embodiments which includes a film former, the film former is a polyacid. Such polyacids are described herein below. For certain of these embodiments, the polyacid is selected from the group consisting of a homopolymer of a monomer, a copolymer of two or more different monomers, and a combination thereof, wherein the monomer and the two or more different monomers are selected from the group consisting of acrylic acid, methacrylic acid, itaconic acid, maleic acid, glutaconic acid, aconitic acid, citraconic acid, mesaconic acid, fumaric acid, and tiglic acid. For certain of these embodiments, the polyacid further comprises a pendent polymerizable group. For certain embodiments, the pendent polymerizable group is preferably an ethylenically unsaturated group. For certain of these embodiments, the ethylenically unsaturated group is a preferably a (meth)acryloyl group and more preferably a methacryloyl group.

In another embodiment, there is provided a remineralizing composition comprising a calcium source, a phosphorous source, and at least one cation selected from the group consisting of cations of Zn, Sn, and Ag, wherein the calcium source, the phosphorous source, and the at least one cation are dissolved in a substantially anhydrous liquid. As indicated above, the cations of tin, zinc, and silver can impart antibacterial properties to the composition, and cations of zinc can further enhance remineralization. For certain embodiments, the remineralizing composition further comprises at least one cation selected from the group consisting of cations of Mg, Ba, and Sr. For certain embodiments, when present, any one or more of these cations is dissolved in the substantially anhydrous liquid. As indicated above, these cations can enhance remineralization, and barium and strontium cations can also impart radiopacity to the composition. For certain embodiments, the remineralizing composition further comprises a second part, wherein the second part comprises an orally acceptable liquid or paste. Orally acceptable liquids and pastes are described herein below.

For certain embodiments, any one of the above compositions further comprises an anticaries agent. Examples of suitable anticaries agents include fluoride sources, organic anticaries agents such as xylitol, organic phosphates (e.g., phytates and glycerophosphates), or a combination of these. For certain embodiments, the anticaries agent is dissolved in the substantially anhydrous liquid. For certain embodiments, preferred fluoride sources include sodium fluoride, stannous fluoride, sodium monofluorophosphate, or combinations thereof.

For certain embodiments, there is provided a remineralizing composition comprising at least one divalent metal cation and a phosphate anion, both of which are dissolved in a substantially anhydrous liquid; wherein the phosphate anion is selected from the group consisting of (P₂O₇)⁻⁴, (H₂PO₂)⁻¹, and an anion represented by the formula: H—[CH(—OR)—]ₓH, wherein x is an integer from 2 to 4; and each R is independently H or —P(O)(O⁻)₂, and wherein at least one R is —P(O)(O⁻)₂. For certain embodiments, the phosphate anion is preferably a glycerophosphate anion (i.e., x is 3 and one R group is —P(O)(O⁻)₂.

It has now been found that certain divalent metal cations and phosphate anions can be separately dissolved in the substantially anhydrous liquid even though the divalent metal cation salts of the phosphate anions have lower solubility or are insoluble at room temperature in the substantially anhydrous liquid.

For certain embodiments of the above compositions containing phosphate anions, the phosphate anion together with the at least one divalent metal cation is a salt which has a lower solubility in the substantially anhydrous liquid than that of either the divalent metal cation or the phosphate anion on a molar basis at 25° C. For certain of these embodiments, the phosphate anion together with the at least one divalent metal cation is a salt which is insoluble in the substantially anhydrous liquid when the salt is combined with the substantially anhydrous liquid. A divalent metal cation salt of a phosphate anion (divalent metal phosphate salt, for example, divalent metal glycerophosphate salt) is insoluble in the substantially anhydrous liquid when the salt dissolves in the liquid at less than 1 percent by weight of the combination of salt and liquid at 25° C. For certain embodiments, the salt is insoluble in the liquid when the salt dissolves in the liquid at less than 0.5 percent, less than 0.2 percent, less than 0.1 percent, less than 0.05 percent, less than 0.01 percent, less than 0.005 percent, or less than 0.001 percent at 25° C. For certain embodiments, the phosphate anion in any one of the above compositions is a glycerophosphate.

Whether a component, such as a salt, is insoluble can be readily determined using known separation techniques, for example, filtration or centrifugation, to determine if there is an insoluble phase present. When present, an insoluble phase will phase separate out of the bulk substance, and can be collected on a filter, such as filter paper, or can be made to settle out using centrifugation. Such separation techniques are conducted after the component has been combined with the substantially anhydrous liquid, but without the presence of a filler or any other component of the composition which is not dissolved in the substantially anhydrous liquid.

In one aspect, the present invention also provides a method of preparing a remineralizing composition comprising dissolving a phosphate anion in a substantially anhydrous liquid to provide a first solution; wherein the phosphate anion is selected from the group consisting of (P₂O₇)⁻⁴, (H₂PO₂)⁻¹, and an anion represented by the formula: H—[CH(—OR)—]ₓH, wherein x is an integer from 2 to 4; and each R is independently H or —P(O)(O⁻)₂, and wherein at least one R is —P(O)(O⁻)₂; dissolving at least one divalent metal cation separately from the phosphate anion in the substantially anhydrous liquid to form a second solution; and combining the first and second solutions. This provides a composition wherein the phosphate anion and the divalent metal cation remain dissolved in the substantially anhydrous liquid. However, if attempts where made to prepare this composition by dissolving the phosphate anion and the divalent metal cation together in the substantially anhydrous liquid, the phosphate anion and the divalent metal cation would be much less soluble compared with the present method or even insoluble. Sources of pyrophosphate anions ((P₂O₇)⁻⁴) which may be dissolved in the substantially anhydrous liquid include, for example, pyrophosphoric acid (H₄P₂O₇) and sodium pyrophosphate hydrate. Sources of hypophosphite anions ((H₂PO₂)⁻¹), which may be dissolved in the substantially anhydrous liquid include, for example, sodium hypophosphite hydrate. Glycerophosphate salts which may be dissolved in the substantially anhydrous liquid, and thereby provide a source of glycerophosphate anions, include, for example, sodium, potassium, and ammonium salts of glycerophosphate. For certain embodiments, the phosphate anion is preferably a glycerophosphate (i.e., x is 3 and one R group is —P(O)(O⁻)₂.

For certain embodiments, including any one of the above embodiments which includes a divalent metal cation and a phosphate anion, the divalent metal cation is preferably selected from the group consisting of divalent cations of Ca, Zn, Sn, Sr, Mg, and Ba. For certain embodiments, the divalent metal cation is preferably Ca⁺².

In another embodiment, there is provided a composition comprising a divalent metal cation source and an organic anticaries agent, both of which are dissolved in a substantially anhydrous liquid, wherein the divalent metal cation of the divalent metal cation source is selected from the group consisting of divalent cations of Ca, Zn, Sr, Ba, and Mg. For certain embodiments, the composition further comprises a phosphorous source dissolved in the substantially anhydrous liquid. For certain of these embodiments, the divalent metal cation source is a salt of the divalent metal cation, wherein the anion of the salt is selected from the group consisting of nitrate, chloride, ethylenediaminetetraacetate, methacrylate, 2-(2-methoxyethoxy)acetate, and a combination thereof.

For certain embodiments, including any one of the above embodiments which includes an organic anticaries agent, the anticaries agent is xylitol.

In another embodiment, there is provided a dental bleach composition comprising a calcium source, a phosphorous source, and a bleaching agent, all of which are dissolved in a substantially anhydrous liquid. For certain embodiments, the bleaching agent is selected from the group consisting of carbamide peroxide, hydrogen peroxide, and a combination thereof. For certain of these embodiments, the bleach composition is a one-part composition.

In another embodiment, there is provided a two-part remineralizing composition comprising a first part comprising a calcium source and a phosphorous source, both of which are dissolved in a substantially anhydrous liquid; and a second part comprising an orally acceptable liquid or paste.

Suitable orally acceptable liquids include, for example, water, any of the substantially anhydrous liquids described herein below, or a combination thereof. The second part, which is provided along with the first part, in certain embodiments, preferably includes an active agent, a polymerizable resin, a film former, or a combination thereof. The active agent may include, for example, an anticaries agent, a bleaching agent, or the like. The orally acceptable paste is a soft, viscous mass comprised of solids dispersed in an orally acceptable liquid. Such solids include, for example, fillers, pigments, dental abrasives, and the like.

For certain embodiments, the first part, the second part, or both the first part and the second part of any one of the two-part remineralizing compositions described above further comprise a thickening agent, a surfactant, or both a thickening agent and a surfactant.

Suitable thickening agents include, for example, carbomers, starch, gum arabic, guar gum, polycaprolactones, poly(N-vinylpyrrolidones), and carboxymethylcellulose. Suitable carbomers include, for example, the CARBOPOL materials (available from Lubrizol Advanced Materials, Inc., Wickliffe, Ohio). When any one of the compositions described herein includes a thickening agent, the amount of thickening agent in the composition is preferably at least 0.01 weight percent, and in some embodiments, at least 0.1, 0.5, 1, 2, 5, or even at least 10 weight percent, based on the total weight of the composition. The amount of thickening agent in the composition is preferably no greater than 20 weight percent, and in some embodiments, not greater than 15, 10, or even no greater than 5 weight percent, based on the total weight of the composition.

Suitable surfactants include, for example, ionic, nonionic, cationic, amphoteric, or combinations thereof. Suitable surfactants may also be polymerizable surfactants. Examples of suitable surfactants are disclosed, for example, in U.S. Pat. Nos. 6,361,761 (Joziak et al.), 5,071,637 (Pellicano), and 5,824,289 (Stoltz). Suitable surfactants include, for example, sodium lauryl sulfate, TOMADOL 45-13 (available from Tomah Reserve Inc., Reserve, La.), and UNITHOX 720 (available from Baker Petrolite Corp., Tulsa, Okla.).

In some embodiments wherein the composition includes a polymer, the polymer can act as the surfactant, for example, when the polymer includes amphoteric segments, such as a quaternary amine segment, or includes the combination of hydrophobic and hydrophilic segments.

When any one of the compositions described herein includes a surfactant, the amount of surfactant in the composition is preferably at least 0.01 weight percent, and in some embodiments at least 0.1, 0.5, 1, 2, 5, or even at least 10 weight percent, based on the total weight of the composition. The amount of surfactant in the composition is preferably no greater than 60 weight percent, and in some embodiments no greater than 50 weight percent or no greater than 20 weight percent, based on the total weight of the composition.

For certain embodiments, including any one of the above embodiments of the two-part remineralizing composition, the first part, second part, or both the first part and the second part further comprise a filler. Suitable fillers are described herein below.

For certain embodiments, including any one of the above embodiments of the two-part remineralizing composition, the second part further comprises a fluoride source. Compounds suitable for use as a fluoride source include, for example, alkali metal fluorides such as sodium fluoride, potassium fluoride, and lithium fluoride; alkali metal monofluorophosphates such as sodium monofluorophosphate, sodium hydrogen monofluorophosphate, and potassium monofluorophosphate; ammonium monofluorophosphate; potassium hexafluorozirconate; potassium hexafluorotitanate; and stannous-containing fluoride compounds such as stannous fluoride and stannous chlorofluoride. These compounds may be used alone or in combination with one another.

Additional examples of compounds that can be used include cesium fluoride, aluminum fluoride, copper fluoride, lead fluoride, iron fluoride, nickel fluoride, zirconium fluoride, silver fluoride, RE fluorides, and amine fluorides such as ammonium fluoride, hexylamine hydrofluoride, lauroylamine hydrofluoride, cetylamine hydrofluoride, glycine hydrofluoride, lysine hydrofluoride, and alanine hydrofluoride. For certain of these embodiments, the fluoride source is selected from the group consisting of stannous fluoride, sodium fluoride, a monofluorophosphate salt, fluoroaluminosilicate glass, a tetrafluoroborate salt, a hexafluorophosphate salt, and a combination thereof.

For certain embodiments, including any one of the above embodiments of the two-part remineralizing composition, the second part further comprises a bleaching agent selected from the group consisting of carbamide peroxide, hydrogen peroxide, a peroxymonophosphate salt, and a combination thereof. For certain embodiments, the bleaching agent is preferably carbamide peroxide.

For certain embodiments, including any one of the above embodiments which include a calcium source, other than a calcium source that includes phosphorous, the calcium source is selected from the group consisting of calcium chloride, calcium nitrate, calcium sulfate, calcium sulfate hemihydrate, calcium sulfate dihydrate, calcium acetate, calcium sodium ethylenediaminetetraacetate, calcium lactate, calcium 2-(2-methoxyethoxy)acetate, calcium methacrylate, calcium phosphoryl choline chloride, calcium laurate, and a combination thereof. For certain embodiments, preferred sources of calcium include calcium chloride, calcium nitrate, calcium acetate, and calcium sodium ethylenediaminetetraacetate.

For certain embodiments, including any one of the above embodiments which include a phosphorus source, other than a phosphorus source that includes calcium, the phosphorous source is selected from the group consisting of phosphorous pentoxide, anhydrous phosphoric acid, a phosphate salt, a phosphate ester, a glycerophosphate salt, a monofluorophosphate salt, a hexafluorophosphate salt, a hypophosphite salt, a phosphonate salt, a pyrophosphate, and a combination thereof. Phosphate salts include, for example, NaH₂PO₄, NaH₂PO₄.2H₂O, NH₄H₂PO₄.2H₂O, K₂HPO₄, KH₂PO₄, or a combination thereof. Phosphate esters include, for example, triphenyl phosphate, triethyl phosphate, diethyl phosphate, and a combination thereof. Glycerophosphate salts include, for example, sodium, potassium, and ammonium salts of glycerophosphate, and a combination thereof. Monofluorophosphate salts include, for example, sodium monofluorophosphate, sodium hydrogen monofluorophosphate, potassium monofluorophosphate, ammonium monofluorophosphate, and a combination thereof. Hexafluorophosphate salts include, for example, sodium hexafluorophosphate, potassium hexafluorophosphate, ammonium hexafluorophosphate, and a combination thereof. Hypophosphite salts include, for example, sodium hypophosphite, potassium hypophosphite, ammonium hypophosphite, hydrates thereof, and a combination thereof. Phosphonate salts include, for example, sodium phosphonate. Pyrophosphates include, for example, pyrophosphoric acid (H₄P₂O₇) and the sodium, potassium, and ammonium pyrophosphate salts.

For certain embodiments, the phosphorous source is selected from the group consisting of phosphorous pentoxide, anhydrous phosphoric acid, sodium dihydrogen phosphate, ammonium dihydrogen phosphate dihydrate, dipotassium hydrogen phosphate, triphenyl phosphate, triethyl phosphate, diethyl phosphate, sodium glycerophosphate, hexafluorophosphate salts of ammonium, sodium, and potassium, sodium monofluorophosphate, sodium hypophosphite, potassium hypophosphite, ammonium hypophosphite, sodium phosphonate, pyrophosphoric acid, the sodium, potassium, and ammonium pyrophosphate salts, and a combination thereof. Whether explicitly stated or not, hydrates of any of the above phosphorous sources are included.

For certain embodiments, including any one of the above embodiments which include a calcium and phosphorous source wherein the source includes calcium and phosphorous, both the calcium source and the phosphorous source are selected from the group consisting of calcium glycerophosphate, calcium phosphoryl choline chloride, and a combination thereof.

In another embodiment, there is provided a remineralizing composition comprising at least one particulate source of calcium and phosphorous combined with a substantially anhydrous liquid, wherein the at least one particulate source is selected from the group consisting of a glass, a glass-ceramic, nanoparticles, nanoclusters, active treated particles, amorphous calcium phosphate, and a combination thereof, and wherein the at least one particulate source includes calcium, phosphorous, or calcium and phosphorous, which can be released; and at least one of 1) a divalent metal salt dissolved in the substantially anhydrous liquid, and 2) a phosphate source dissolved in the substantially anhydrous liquid.

Suitable particulate sources of calcium and phosphorous are described herein below.

Suitable divalent metal salts include, for example, a combination of at least one divalent cation of Ca, Zn, Sn, Sr, Mg, or Ba in combination with at least one anion selected from the group consisting of nitrate, chloride, ethylenediaminetetraacetate, methacrylate, and 2-(2-methoxyethoxy)acetate. Suitable phosphate sources include, for example, NaH₂PO₄, NaH₂PO₄.2H₂O, NH₄H₂PO₄.2H₂O, K₂HPO₄, KH₂PO₄, triphenyl phosphate, triethyl phosphate, diethyl phosphate, sodium glycerophosphate, ammonium glycerophosphate, potassium glycerophosphate, pyrophosphoric acid (H₄P₂O₇), sodium pyrophosphate, potassium pyrophosphate, ammonium pyrophosphate, sodium hexafluorophosphate, potassium hexafluorophosphate, ammonium hexafluorophosphate, sodium monofluorophosphate, or a combination thereof.

For certain embodiments, including anyone of the above embodiments of compositions except those which already include a fluoride source, the composition further includes a fluoride source dissolved in the substantially anhydrous liquid. For certain of these embodiments, the fluoride source is selected from the group consisting of stannous fluoride, stannous chlorofluoride, sodium fluoride, a monofluorophosphate salt (e.g., sodium monofluorophosphate, sodium hydrogen monofluorophosphate, potassium monofluorophosphate, ammonium monofluorophosphate), a hexafluorophosphate salt (e.g., sodium hexafluorophosphate, ammonium hexafluorophosphate), a tetrafluoroborate salt, and a combination thereof.

Tetrafluoroborate salts include those described in U.S. Pat. No. 4,871,786 (Aasen), which is incorporated herein by reference, and referred to therein as organic fluoride sources, which include a quaternary ammonium, iodonium, sulfonium, or phosphonium cation.

For certain embodiments, including anyone of the above embodiments of compositions except those which already include a fluoride source, the composition further includes a fluoride source dispersed in the substantially anhydrous liquid. Suitable fluoride sources include, for example, fluoroaluminosilicate glass, metallofluorocomplexes, fluoride salts, amine fluorides, potassium hexafluorozirconate and potassium hexafluorotitanate. For certain of these embodiments, the fluoride source is selected from the group consisting of fluoroaluminosilicate glass, metallofluorocomplexes, fluoride salts, and amine fluorides. Suitable metallofluorocomplexes are described in U.S. Pat. No. 6,391,286 (Mitra et al.), which is incorporated herein by reference. Suitable fluoride salts include, for example, barium fluoride, calcium fluoride, magnesium fluoride, potassium fluoride, sodium fluoride, lithium fluoride, strontium fluoride, RE fluorides, cesium fluoride, aluminum fluoride, copper fluoride, lead fluoride, iron fluoride, nickel fluoride, zirconium fluoride, and silver fluoride. Suitable amine fluorides include, for example, ammonium fluoride, ammonium hydrogen difluoride, hexylamine hydrofluoride, lauroylamine hydrofluoride, cetylamine hydrofluoride, N-[N,N-bis(2-hydroxyethyl)aminopropyl]-N-(2-hydroxethyl)octadecylamine dihydrofluoride, glycine hydrofluoride, alanine hydrofluoride, and lysine hydrofluoride.

For certain embodiments, including any one of the above embodiments except those which include a polymerizable resin, the composition further includes a polymerizable resin.

For certain embodiments, including any one of the above embodiments of a two-part remineralizing composition which includes a polymerizable resin, the first part, the second part, or both the first part and the second part comprise the polymerizable resin.

For certain embodiments, including any one of the above embodiments which include a polymerizable resin, the polymerizable resin is dissolved in the substantially anhydrous liquid, wherein the polymerizable resin optionally comprises a portion of the substantially anhydrous liquid.

Alternatively, for certain embodiments, including any one of the above embodiments which include a polymerizable resin, the substantially anhydrous liquid is the polymerizable resin.

For certain embodiments, including any one of the above embodiments which include a polymerizable resin, the polymerizable resin is selected from the group consisting of an ethylenically unsaturated compound with acid functionality, an ethylenically unsaturated compound without acid functionality, an oxirane, a silane, and a combination thereof. For certain of these embodiments, the polymerizable resin is selected from the group consisting of an ethylenically unsaturated compound with acid functionality, an ethylenically unsaturated compound without acid functionality, and a combination thereof. For certain of these embodiments, the acid functionality is selected from the group consisting of carboxylic acid functionality, phosphoric acid functionality, phosphonic acid functionality, sulfonic acid functionality, and a combination thereof. Alternatively, for certain of these embodiments, the polymerizable resin comprises a silane, wherein the silane includes at least one of a silane monomer, a silane oligomer, and a silane polymer.

Suitable substantially anhydrous liquids include those in which the calcium source, phosphorous source, phosphate salts, phosphate anions, metal salts, and or metal cations as described above are soluble. For certain embodiments, any one of these components has a solubility of at least 0.01, 0.05, 0.1, 0.2, 0.5, 1, 2, 5, or at least 10% by weight in the substantially anhydrous liquid. For certain embodiments, the calcium source has a solubility of at least 0.01, 0.05, 0.1, 0.2, 0.5, 1, 2, 5, or at least 10% by weight in the substantially anhydrous liquid. For certain of these embodiments, the phosphorous source has a solubility of at least 0.01, 0.05, 0.1, 0.2, 0.5, 1, 2, 5, or at least 10% by weight in the substantially anhydrous liquid.

For certain embodiments, including any one of the above embodiments except those wherein the polymerizable resin is the substantially anhydrous liquid, the substantially anhydrous liquid is selected from the group consisting of ethanol, triethanolamine, methoxypropanol, isopropanol, ethyl acetate, glycerol, poly(ethylene glycol), propylene glycol, poly(propylene glycol), hydroxyethyl methacrylate, poly(ethylene glycol) dimethacrylate, hydroxyethyl methacrylate phosphate, methacryloyloxyhexyl phosphate, methacryloyloxydecyl phosphate, glycerol dimethacrylate phosphate, citric dimethacrylate, propionic dimethacrylate, an oxirane, a silane polymer, and a combination thereof. For certain of these embodiments, the substantially anhydrous liquid is selected from the group consisting of ethanol, triethanolamine, methoxypropanol, isopropanol, ethyl acetate, glycerol, poly(ethylene glycol), propylene glycol, and poly(propylene glycol).

For certain embodiments, including any one of the above embodiments wherein the polymerizable resin is the substantially anhydrous liquid, the substantially anhydrous liquid is selected from the group consisting of hydroxyethyl methacrylate, poly(ethylene glycol) dimethacrylate, hydroxyethyl methacrylate phosphate, methacryloyloxyhexyl phosphate, methacryloyloxydecyl phosphate, glycerol dimethacrylate phosphate, citric dimethacrylate, propionic dimethacrylate, an oxirane, a silane polymer, and a combination thereof.

For certain embodiments, including any one of the above composition embodiments, the composition is for contacting a tooth structure.

For certain embodiments, including any one of the above embodiments which includes a polymerizable resin or any one of the above embodiments which includes a film former having a polymerizable group, the composition is selected from the group consisting of a restorative, a glass ionomer restorative, a dental primer, a dental adhesive, a cavity liner, a cavity cleansing agent, a cement, a glass ionomer cement, a dental cement (temporary or permanent), a varnish, a dental coating, an orthodontic adhesive, an orthodontic primer, an orthodontic cement, an endodontic filling material, a pit and fissure sealant, and a desensitizer.

For certain embodiments, including any one of the above embodiments except those which include a polymerizable resin or a film former having a polymerizable group, the composition is selected from the group consisting of a sealant, a desensitizer, an enamel conditioning material, a prophy paste, an ion recharge paste or gel, a mousse, a spray, a rinse, a rinse concentrate, a mouthwash, a whitening composition, a dentifrice, a coating, a varnish, an adhesive strip, a foam, a cavity cleansing agent, a dental primer, and a cavity liner.

In one embodiment, the present invention provides a kit comprising any one of the above compositions and an applicator. For certain of these embodiments, the applicator is selected from the group consisting of a container, a sprayer, a brush, a swab, a tray, and a combination thereof. For certain of these embodiments, the kit further comprises a material selected from the group consisting of orthodontic brackets, orthodontic appliances, restoratives, dental prostheses, dental implants, dental appliances, dental primers, dental adhesives, cavity liners, cavity cleansing agents, varnishes, glass ionomers, orthodontic adhesives, orthodontic primers, orthodontic cements, cements, sealants, desensitizers, enamel conditioning materials, prophy pastes, ion recharge pastes or gels, rinses, rinse concentrates, mouth washes, whitening compositions, dentifrices, coatings, adhesive strips, foams, and combinations thereof.

### Polymerizable Resins

Compositions of the present invention which include a polymerizable resin may be used for treating hard surfaces, preferably, tooth structures such as dentin and enamel, and bone. These compositions can be used as described below, for example, as dental materials and dental adhesives. In some embodiments, the compositions can be hardened (e.g., polymerized by conventional photopolymerization and/or chemical polymerization techniques) prior to applying a dental material. In other embodiments, the compositions can be hardened after applying a dental material.

Polymerizable resins that are photopolymerizable and thereby render the composition photopolymerizable include ethylenically unsaturated compounds (which contain free radically active unsaturated groups, e.g., acrylates and methacrylates), oxiranes, generally known as epoxy resins (which contain cationically active oxirane rings), vinyl ether resins (which contain cationically active vinyl ether groups), and combinations thereof. Polymerizable resins can contain both a cationically active functional group and a free radically active functional group in a single compound. Examples include epoxy-functional (meth)acrylates.

Ethylenically unsaturated compounds include monomers, oligomers, and polymers having ethylenic unsaturation and can further have acid functionality and/or acid-precursor functionality. Acid functionality includes, for example, carboxylic acid functionality, phosphoric acid functionality, phosphonic acid functionality, sulfonic acid functionality, and combinations thereof. Acid-precursor functionalities include, for example, anhydrides, acid halides, and pyrophosphates.

Ethylenically unsaturated compounds with acid functionality include, for example, α,β-unsaturated acidic compounds such as glycerol phosphate mono(meth)acrylates, glycerol phosphate di(meth)acrylates, hydroxyethyl (meth)acrylate (e.g., HEMA) phosphates, bis((meth)acryloxyethyl)phosphate, ((meth)acryloxypropyl)phosphate, bis((meth)acryloxypropyl)phosphate, bis((meth)acryloxy)propyloxy phosphate, (meth)acryloxyhexyl phosphate, bis((meth)acryloxyhexyl)phosphate, (meth)acryloxyoctyl phosphate, bis((meth)acryloxyoctyl)phosphate, (meth)acryloxydecyl phosphate, bis((meth)acryloxydecyl)phosphate, caprolactone methacrylate phosphate, citric acid di- or tri-methacrylates, poly(meth)acrylated oligomaleic acid, poly(meth)acrylated polymaleic acid, poly(meth)acrylated poly(meth)acrylic acid, poly(meth)acrylated polycarboxyl-polyphosphonic acid, poly(meth)acrylated polychlorophosphoric acid, poly(meth)acrylated polysulfonate, 2-sulfoethyl methacrylate, 3-sulfopropyl methacrylate, 2-acrylamido 2-methylpropane sulfonate, poly(meth)acrylated polyboric acid, and the like. Monomers, oligomers, and polymers of unsaturated carbonic acids such as (meth)acrylic acids, aromatic (meth)acrylated acids (e.g., methacrylated trimellitic acids), and anhydrides are also included. For certain embodiments, preferred ethylenically unsaturated compounds with acid functionality include hydroxyethyl methacrylate phosphate, methacryloyloxyhexyl phosphate, methacryloyloxydecyl phosphate, glycerol dimethacrylate phosphate, citric dimethacrylate, and propionic dimethacrylate,

Certain of these compounds are obtained, for example, as reaction products between isocyanatoalkyl (meth)acrylates and carboxylic acids. Additional compounds of this type having both acid-functionality and ethylenically unsaturated components are described in U.S. Pat. Nos. 4,872,936 (Engelbrecht) and 5,130,347 (Mitra). A wide variety of such compounds containing both the ethylenically unsaturated and acid moieties can be used. Mixtures of such compounds can be used if desired. Additional ethylenically unsaturated compounds with acid functionality include, for example, AA:ITA:IEM (copolymer of acrylic acid:itaconic acid with pendent methacrylate made by reacting AA:ITA copolymer with sufficient 2-isocyanatoethyl methacrylate to convert a portion of the acid groups of the copolymer to pendent methacrylate groups as described, for example, in Example 11 of U.S. Pat. No. 5,130,347 (Mitra)); and those recited in U.S. Pat. Nos. 4,259,075 (Yamauchi et al.), 4,499,251 (Omura et al.), 4,537,940 (Omura et al.), 4,539,382 (Omura et al.), 5,530,038 (Yamamoto et al.), 6,458,868 (Okada et al.), and European Pat. Application Publication Nos. EP 712,622 (Tokuyama Corp.) and EP 1,051,961 (Kuraray Co., Ltd.).

For certain embodiments, preferably, the compositions of the present invention which include a polymerizable resin include at least 1% by weight, more preferably at least 3% by weight, and most preferably at least 5% by weight ethylenically unsaturated compounds with acid functionality, based on the total weight of the unfilled composition. Preferably, compositions of the present invention include at most 80% by weight, more preferably at most 70% by weight, and most preferably at most 60% by weight ethylenically unsaturated compounds with acid functionality, based on the total weight of the unfilled composition.

The compositions of the present invention which include a polymerizable resin may include one or more ethylenically unsaturated compounds without acid functionality instead of or in addition to the ethylenically unsaturated compounds with acid functionality, thereby forming hardenable compositions. These polymerizable resins may be monomers, oligomers, or polymers.

For certain embodiments, preferably, compositions of the present invention include at least 5% by weight, more preferably at least 10% by weight, and most preferably at least 15% by weight ethylenically unsaturated compounds without acid functionality, based on the total weight of the unfilled composition. Preferably, compositions of the present invention include at most 95% by weight, more preferably at most 90% by weight, and most preferably at most 80% by weight ethylenically unsaturated compounds without acid functionality, based on the total weight of the unfilled composition.

In certain embodiments, the compositions which include a polymerizable resin are photopolymerizable, i.e., the compositions contain a photopolymerizable resin and a photoinitiator (e.g., a photoinitiator system) that upon irradiation with actinic radiation initiates the polymerization (or hardening) of the composition. Such photopolymerizable compositions can be free radically polymerizable.

In certain embodiments, the compositions which include a polymerizable resin are chemically polymerizable, i.e., the compositions contain a chemically polymerizable resin and a chemical initiator (e.g., initiator system) that can polymerize, cure, or otherwise harden the composition without dependence on irradiation with actinic radiation. Such chemically polymerizable compositions are sometimes referred to as “self-cure” compositions and may include glass ionomer cements, resin-modified glass ionomer cements, redox cure systems, and combinations thereof.

Suitable photopolymerizable resins include ethylenically unsaturated compounds (which contain free radically active unsaturated groups). Examples of useful ethylenically unsaturated compounds include acrylic acid esters, methacrylic acid esters, hydroxy-functional acrylic acid esters, hydroxy-functional methacrylic acid esters, and combinations thereof.

Photopolymerizable resins may include compounds having free radically active functional groups, and may include monomers, oligomers, and polymers having one or more ethylenically unsaturated group. Suitable compounds contain at least one ethylenically unsaturated bond and are capable of undergoing addition polymerization. Such free radically polymerizable compounds include mono-, di- or poly-(meth)acrylates (i.e., acrylates and methacrylates) such as, methyl (meth)acrylate, ethyl acrylate, isopropyl methacrylate, n-hexyl acrylate, stearyl acrylate, allyl acrylate, glycerol triacrylate, ethyleneglycol diacrylate, diethyleneglycol diacrylate, triethyleneglycol dimethacrylate, 1,3-propanediol di(meth)acrylate, trimethylolpropane triacrylate, 1,2,4-butanetriol trimethacrylate, 1,4-cyclohexanediol diacrylate, pentaerythritol tetra(meth)acrylate, sorbitol hexacrylate, tetrahydrofurfuryl (meth)acrylate, bis[1-(2-acryloxy)]-p-ethoxyphenyldimethylmethane, bis[1-(3-acryloxy-2-hydroxy)]-p-propoxyphenyldimethylmethane, ethoxylated bisphenolA di(meth)acrylate, and trishydroxyethyl-isocyanurate trimethacrylate; (meth)acrylamides (i.e., acrylamides and methacrylamides) such as (meth)acrylamide, methylene bis-(meth)acrylamide, and diacetone (meth)acrylamide; urethane (meth)acrylates; the bis-(meth)acrylates of polyethylene glycols (preferably of molecular weight 200-500), copolymerizable mixtures of acrylated monomers such as those in U.S. Pat. No. 4,652,274 (Boettcher et al.), acrylated oligomers such as those of U.S. Pat. No. 4,642,126 (Zador et al.), and poly(ethylenically unsaturated) carbamoyl isocyanurates such as those disclosed in U.S. Pat. No. 4,648,843 (Mitra); and vinyl compounds such as styrene, diallyl phthalate, divinyl succinate, divinyl adipate and divinyl phthalate. Other suitable free radically polymerizable compounds include siloxane-functional (meth)acrylates as disclosed, for example, in WO 00/38619 (Guggenberger et al.), WO 01/92271 (Weinmann et al.), WO 01/07444 (Guggenberger et al.), WO 00/42092 (Guggenberger et al.) and fluoropolymer-functional (meth)acrylates as disclosed, for example, in U.S. Pat. No. 5,076,844 (Fock et al.), U.S. Pat. No. 4,356,296 (Griffith et al.), EP 0 373 384 (Wagenknecht et al.), EP 0 201 031 (Reiners et al.), and EP 0 201 778 (Reiners et al.). Mixtures of two or more free radically polymerizable compounds can be used if desired.

The polymerizable resin may also contain hydroxyl groups and free radically active functional groups in a single molecule. Examples of such materials include hydroxyalkyl (meth)acrylates, such as 2-hydroxyethyl (meth)acrylate and 2-hydroxypropyl (meth)acrylate; glycerol mono- or di-(meth)acrylate; trimethylolpropane mono- or di-(meth)acrylate; pentaerythritol mono-, di-, and tri-(meth)acrylate; sorbitol mono-, di-, tri-, tetra-, or penta-(meth)acrylate; and 2,2-bis[4-(2-hydroxy-3-methacryloxypropoxy)phenyl]propane (bisGMA). Suitable ethylenically unsaturated compounds are also available from a wide variety of commercial sources, such as Sigma-Aldrich, St. Louis. Mixtures of ethylenically unsaturated compounds can be used if desired.

Photopolymerizable resins may also include PEGDMA (polyethyleneglycol dimethacrylate having a molecular weight of approximately 400), bisGMA, UDMA (urethane dimethacrylate), GDMA (glycerol dimethacrylate), TEGDMA (triethyleneglycol dimethacrylate), bisEMA6 as described in U.S. Pat. No. 6,030,606 (Holmes), and NPGDMA (neopentylglycol dimethacrylate). Various combinations of the polymerizable resins can be used if desired.

For certain embodiments, preferred polymerizable resins, which are photopolymerizable ethylenically unsaturated compounds without acid functionality include hydroxyethyl methacrylate and poly(ethylene glycol) dimethacrylate.

Oxiranes which are suitable for use as polymerizable resins in the present compositions include, for example, cycloaliphatic oxiranes, aliphatic oxiranes, aromatic oxiranes, or a combination thereof. These compounds, which are widely known as epoxy compounds, can be monomeric, polymeric, or mixtures thereof. These materials generally have, on the average, at least one polymerizable epoxy group (oxirane unit) per molecule, and preferably at least about 1.5 polymerizable epoxy groups per molecule. The polymeric epoxides include linear polymers having terminal epoxy groups (e.g., a diglycidyl ether of a polyoxyalkylene glycol), polymers having skeletal oxirane units (e.g., polybutadiene polyepoxide), and polymers having pendent epoxy groups (e.g., a glycidyl methacrylate polymer or copolymer). The epoxides may be pure compounds or may be mixtures containing one, two, or more epoxy groups per molecule. The “average” number of epoxy groups per molecule is determined by dividing the total number of epoxy groups in epoxy-containing material by the total number of epoxy molecules present. The epoxy compounds may have a molecular weight of from about 58 to about 100,000 or more. The epoxy compounds may further include substituent groups that do not substantially interfere with cationic cure at room temperature, such as halogens, ester groups, ethers, sulfonate groups, siloxane groups, nitro groups, phosphate groups, and the like. Suitable oxiranes include those which contain cyclohexene oxide groups, such as the epoxycyclohexanecarboxylates, for example, 3,4-epoxycyclohexylmethyl-3,4-epoxycyclohexanecarboxylate, 3,4-epoxy-2-methylcyclohexylmethyl-3,4-epoxy-2-methylcyclohexane carboxylate, and bis(3,4-epoxy-6-methylcyclohexylmethyl)adipate. A more detailed list of useful epoxides of this nature is provided in U.S. Pat. No. 3,117,099 (Proops et al.), which is incorporated herein by reference.

Suitable oxiranes also include glycidyl ether compounds, such as glycidoxyalkyl and glycidoxyaryl compounds containing 1 to 6 glycidoxy groups. Examples include glycidyl ethers of polyhydric phenols, which can be obtained by reacting the polyhydric phenol with an excess of epichlorohydrin to provide, for example, 2,2-bis(2,3-epoxypropoxyphenyl)propane. Additional epoxides of this type are described in U.S. Pat. No. 3,018,262 (Schroeder), which is incorporated herein by reference, and in “Handbook of Epoxy Resins” by Lee and Neville, McGraw-hill Book Co., New York (1967).

Many suitable oxiranes are commerically available and are listed in U.S. Pat. No. 6,187,833 (Oxman et al.).

Silanes which are suitable polymerizable resins for use in the present compositions include, for example, methacryloxyalkyltrimethoxysilanes (available under the trade designation WACKER SILANE GF 31 from Wacker Silicones, Munich, Germany), gamma-methacryloxypropyltrimethoxysilane, styrylethyltrimethoxysilane (available from Gelest Inc., Tullytown, Pa.), gamma-glycidoxypropyltrimethoxysilane, and poly(alkylene oxide) group-containing silanes such as gamma-[poly(alkylene oxide)]propyltrimethoxysilane described in U.S. Publication No. 2004/010055, which is incorporated herein by reference. Suitable silanes may also include such groups as alkyl, hydroxyalkyl, hydroxyaryl, and aminoalkyl groups.

Suitable silanes also include silane oligomers. In one example, the silane oligomer is an ethylenically unsaturated preformed organosiloxane chain of the formula X(Y)ₙSi(R′)₃₋ₘZₘ, wherein X is a vinyl group; Y is a divalent linking group (e.g., alkylene, arylene, alkarylene, or aralkylene of 1 to 30 carbon atoms) which may include heteroatoms (e.g., O, N, S, P) as in ester, amide, urethane, and urea groups; n is 0 or 1; m is an integer from 1 to 3; R′ is hydrogen, C₁₋₄ alkyl (methyl, ethyl, propyl), C₆₋₂₀ aryl (e.g., phenyl), or C₁₋₄ alkoxy; and Z is a monovalent siloxane polymeric moiety having a number average molecular weight above about 500 and essentially unreactive under copolymerization conditions. These silane oligomers are described in U.S. Pat. No. 6,596,403 (Mitra et al.), which is incorporated herein by reference. In another example, the silane oligomer can be an organopolysiloxane with at least two ethylenically unsaturated groups, an organohydrogenpolysiloxane with at least three Si—H groups, a silane dendrimer with terminal alkenyl groups, or a combination thereof. These silane oligomers are described in U.S. Pat. Nos. 6,335,413 (Zech et al.) and 6,566,413 (Weinmann et al.), which are incorporated herein by reference. In another example, the silane oligomer can be a polyhedral oligomeric silsesquioxane of the generic formula (R″SiO_{1.5})_{n′}, wherein R″ is a hydrocarbon and n′ is 6, 8, 10, 12, or higher, wherein one or more of the hydrocarbon groups are replaced or functionalized with an acrylate- or methacrylate-containing group (e.g., methacryloxypropyl). These compounds (available from Gelest, Inc., Tullytown, Pa.; and Hybrid Plastics, Inc. under the trade name POSS NANOSTRUCTURED CHEMICALS) are described in U.S. Pat. No. 6,653,365, which is incorporated herein by reference.

Suitable photoinitiators (i.e., photoinitiator systems that include one or more compounds) for polymerizing free radically photopolymerizable resins include binary and tertiary systems. Typical tertiary photoinitiators include an iodonium salt, a photosensitizer, and an electron donor compound as described in U.S. Pat. No. 5,545,676 (Palazzotto et al.). Preferred iodonium salts are the diaryl iodonium salts, e.g., diphenyliodonium chloride, diphenyliodonium hexafluorophosphate, diphenyliodonium tetrafluoroborate, and tolylcumyliodonium tetrakis(pentafluorophenyl)borate. Preferred photosensitizers are monoketones and diketones that absorb some light within a range of 400 nm to 520 nm (preferably, 450 nm to 500 nm). More preferred compounds are alpha diketones that have some light absorption within a range of 400 nm to 520 nm (even more preferably, 450 to 500 nm). Preferred compounds are camphorquinone, benzil, furil, 3,3,6,6-tetramethylcyclohexanedione, phenanthraquinone, 1-phenyl-1,2-propanedione and other 1-aryl-2-alkyl-1,2-ethanediones, and cyclic alpha diketones. Most preferred is camphorquinone. Preferred electron donor compounds include substituted amines, e.g., ethyl dimethylaminobenzoate. Other suitable tertiary photoinitiator systems useful for photopolymerizing cationically polymerizable resins are described, for example, in U.S. Pat. Publication No. 2003/0166737 (Dede et al.).

Other suitable photoinitiators for polymerizing free radically photopolymerizable compositions include the class of phosphine oxides that typically have a functional wavelength range of 380 nm to 1200 nm. Preferred phosphine oxide free radical initiators with a functional wavelength range of 380 nm to 450 nm are acyl and bisacyl phosphine oxides such as those described in U.S. Pat. Nos. 4,298,738 (Lechtken et al.), 4,324,744 (Lechtken et al.), 4,385,109 (Lechtken et al.), 4,710,523 (Lechtken et al.), and 4,737,593 (Ellrich et al.), 6,251,963 (Kohler et al.); and EP Application No. 0 173 567 A2 (Ying).

Commercially available phosphine oxide photoinitiators capable of free-radical initiation when irradiated at wavelength ranges of greater than 380 nm to 450 nm include bis(2,4,6-trimethylbenzoyl)phenyl phosphine oxide (IRGACURE 819, Ciba Specialty Chemicals, Tarrytown, N.Y.), bis(2,6-dimethoxybenzoyl)-(2,4,4-trimethylpentyl) phosphine oxide (CGI 403, Ciba Specialty Chemicals), a 25:75 mixture, by weight, of bis(2,6-dimethoxybenzoyl)-2,4,4-trimethylpentyl phosphine oxide and 2-hydroxy-2-methyl-1-phenylpropan-1-one (IRGACURE 1700, Ciba Specialty Chemicals), a 1:1 mixture, by weight, of bis(2,4,6-trimethylbenzoyl)phenyl phosphine oxide and 2-hydroxy-2-methyl-1-phenylpropane-1-one (DAROCUR 4265, Ciba Specialty Chemicals), and ethyl 2,4,6-trimethylbenzylphenyl phosphinate (LUCIRIN LR8893X, BASF Corp., Charlotte, N.C.).

Typically, the phosphine oxide initiator is present in the photopolymerizable composition in catalytically effective amounts, such as from 0.1 weight percent to 5.0 weight percent, based on the total weight of the composition.

Tertiary amine reducing agents may be used in combination with an acylphosphine oxide. Illustrative tertiary amines useful in the invention include ethyl 4-(N,N-dimethylamino)benzoate and N,N-dimethylaminoethyl methacrylate. When present, the amine reducing agent is present in the photopolymerizable composition in an amount from 0.1 weight percent to 5.0 weight percent, based on the total weight of the composition. Useful amounts of other initiators are well known to those of skill in the art.

Suitable chemically polymerizable resins may be polymerized using a redox cure system. A polymerizable resin (e.g., an ethylenically unsaturated polymerizable resin) may be combined with redox agents that include an oxidizing agent and a reducing agent. Suitable polymerizable resins, redox agents, optional acid-functional components, and optional fillers that may be used are described in U.S. Pat. Publication Nos. 2003/0166740 (Mitra et al.) and 2003/0195273 (Mitra et al.).

The reducing and oxidizing agents should react with or otherwise cooperate with one another to produce free-radicals capable of initiating polymerization of the resin (e.g., the ethylenically unsaturated resin). This type of cure is a dark reaction, that is, it is not dependent on the presence of light and can proceed in the absence of light. The reducing and oxidizing agents are preferably sufficiently shelf-stable and free of undesirable colorization to permit their storage and use under typical dental conditions. They should be sufficiently miscible with the resin system to permit ready dissolution in (and discourage separation from) the other components of the polymerizable composition.

Useful reducing agents include ascorbic acid, ascorbic acid derivatives, and metal complexed ascorbic acid compounds as described in U.S. Pat. No. 5,501,727 (Wang et al.); amines, especially tertiary amines, such as 4-tert-butyl dimethylaniline; aromatic sulfinic salts, such as p-toluenesulfinic salts and benzenesulfinic salts; thioureas, such as 1-ethyl-2-thiourea, tetraethyl thiourea, tetramethyl thiourea, 1,1-dibutyl thiourea, and 1,3-dibutyl thiourea; and mixtures thereof. Other secondary reducing agents may include cobalt (II) chloride, ferrous chloride, ferrous sulfate, hydrazine, hydroxylamine (depending on the choice of oxidizing agent), salts of a dithionite or sulfite anion, and mixtures thereof. Preferably, the reducing agent is an amine.

Suitable oxidizing agents will also be familiar to those skilled in the art, and include but are not limited to persulfuric acid and salts thereof, such as sodium, potassium, ammonium, cesium, and alkyl ammonium salts. Additional oxidizing agents include peroxides such as benzoyl peroxides, hydroperoxides such as cumyl hydroperoxide, t-butyl hydroperoxide, and amyl hydroperoxide, as well as salts of transition metals such as cobalt (III) chloride and ferric chloride, cerium (IV) sulfate, perboric acid and salts thereof, permanganic acid and salts thereof, perphosphoric acid and salts thereof, and mixtures thereof.

It may be desirable to use more than one oxidizing agent or more than one reducing agent. Small quantities of transition metal compounds may also be added to accelerate the rate of redox cure. In some embodiments it may be preferred to include a secondary ionic salt to enhance the stability of the polymerizable composition as described in U.S. Pat. Publication No. 2003/0195273 (Mitra et al.).

The reducing and oxidizing agents are present in amounts sufficient to permit an adequate free-radical reaction rate. This can be evaluated by combining all of the ingredients of the polymerizable composition except for the optional filler, and observing whether or not a hardened mass is obtained.

Preferably, the reducing agent is present in an amount of at least 0.01% by weight, and more preferably at least 0.1% by weight, based on the total weight of the components of the polymerizable composition. Preferably, the reducing agent is present in an amount of no greater than 10% by weight, and more preferably no greater than 5% by weight, based on the total weight of the components of the polymerizable composition.

Preferably, the oxidizing agent is present in an amount of at least 0.01% by weight, and more preferably at least 0.10% by weight, based on the total weight of the components of the polymerizable composition. Preferably, the oxidizing agent is present in an amount of no greater than 10% by weight, and more preferably no greater than 5% by weight, based on the total weight of the components of the polymerizable composition.

The reducing or oxidizing agents can be microencapsulated as described in U.S. Pat. No. 5,154,762 (Mitra et al.). This will generally enhance shelf stability of the polymerizable composition, and if necessary permit packaging the reducing and oxidizing agents together. For example, through appropriate selection of an encapsulant, the oxidizing and reducing agents can be combined with an acid-functional component and optional filler and kept in a storage-stable state.

A redox cure system can be combined with other cure systems, e.g., with a photopolymerizable composition such as described U.S. Pat. No. 5,154,762 (Mitra et al.).

In some embodiments, compositions of the present invention which include a polymerizable resin can be hardened to fabricate a dental article selected from the group consisting of crowns, fillings, mill blanks, orthodontic devices, and prostheses.

### Film Formers

Compositions of the present invention which include a film former can be used as described below, for example, as coatings, varnishes, sealants, primers, and desensitizers. Film formers include polymers with a repeating unit that includes a polar or polarizable group as described herein below. In certain embodiments, the film formers also include a repeating unit that includes a fluoride releasing group, a repeating unit that includes a hydrophobic hydrocarbon group, a repeating unit that includes a graft polysiloxane chain, a repeating unit that includes a hydrophobic fluorine-containing group, a repeating unit that includes a modulating group, or a combination thereof, as described herein below. In certain embodiments, the film former optionally includes a pendent polymerizable group (e.g., ethylenically unsaturated groups, epoxy groups, or silane moieties capable of undergoing a condensation reaction). Exemplary film formers are disclosed, for example, in U.S. Pat. Nos. 5,468,477 (Kumar et al.), 5,525,648 (Aasen et al.), 5,607,663 (Rozzi et al.), 5,662,887 (Rozzi et al.), 5,725,882 (Kumar et al.), 5,866,630 (Mitra et al.), 5,876,208 (Mitra et al.), 5,888,491 (Mitra et al.), and 6,312,668 (Mitra et al.).

Repeating units including a polar or polarizable group are derived from vinylic monomers such as acrylates, methacrylates, crotonates, itaconates, and the like. The polar groups can be acidic, basic or salt. These groups can also be ionic or neutral.

Examples of polar or polarizable groups include neutral groups such as hydroxy, thio, substituted and unsubstituted amido, cyclic ethers (such as oxanes, oxetanes, furans and pyrans), basic groups (such as phosphines and amines, including primary, secondary, tertiary amines), acidic groups (such as oxy acids, and thiooxyacids of C, S, P, B), ionic groups (such as quaternary ammonium, carboxylate salt, sulfonic acid salt and the like), and the precursors and protected forms of these groups. Additionally, a polar or polarizable group could be a macromonomer. More specific examples of such groups follow.

Polar or polarizable groups may be derived from mono- or multifunctional carboxyl group containing molecules represented by the general formula:

CH₂═CR²G-(COOH)_{d}

where R² is H, methyl, ethyl, cyano, carboxy, or carboxymethyl, d is an integer from 1 to 5 and G is a bond or a hydrocarbyl radical linking group containing from 1 to 12 carbon atoms of valence d+1 and optionally substituted with and/or interrupted with a substituted or unsubstituted heteroatom (such as O, S, N and P). Optionally, this unit may be provided in its salt form. The polymers containing repeating units resulting from polymerization of these monomers are polyacids. For certain embodiments, preferred monomers in this class include acrylic acid, methacrylic acid, itaconic acid, maleic acid, glutaconic acid, aconitic acid, citraconic acid, mesaconic acid, fumaric acid, and tiglic acid. For certain embodiments, polyacids used in the present compositions are homopolymers and/or copolymers of these monomers.

Polar or polarizable groups may, for example, be derived from mono- or multifunctional hydroxy group containing molecules represented by the general formula:

CH₂═CR²—CO-L-R³—(OH)_{d}

where R² is H, methyl, ethyl, cyano, carboxy, or carboxyalkyl, L is O or NH, d is an integer from 1 to 5 and R³ is a hydrocarbyl radical of valence d+1 containing from 1-12 carbon atoms. For certain embodiments, preferred monomers in this class include hydroxyethyl (meth)acrylate, hydroxypropyl (meth)acrylate, hydroxybutyl (meth)acrylate, glycerol mono(meth)acrylate, tris(hydroxymethyl)ethane monoacrylate, pentaerythritol mono(meth)acrylate, N-hydroxymethyl (meth)acrylamide, hydroxyethyl (meth)acrylamide, and hydroxypropyl (meth)acrylamide.

Polar or polarizable groups may alternatively be derived from mono- or multifunctional amino group containing molecules of the general formula:

CH₂═CR²—CO-L-R³—(NR⁴R⁵)_{d}

where R², L, R³, and d are as defined above and R⁴ and R⁵ are independently H or alkyl groups of 1 to 12 carbon atoms or together they constitute a heterocyclic group. Preferred monomers of this class are aminoethyl (meth)acrylate, aminopropyl (meth)acrylate, N,N-dimethylaminoethyl (meth)acrylate, N,N-diethylaminoethyl (meth)acrylate, N,N-dimethylaminopropyl (meth)acrylamide, N-isopropylaminopropyl (meth)acrylamide, and 4-methyl-1-acryloyl-piperazine.

Polar or polarizable groups may also be derived from alkoxy substituted (meth)acrylates or (meth)acrylamides, such as methoxyethyl (meth)acrylate, 2-(2-ethoxyethoxy)ethyl (meth)acrylate, polyethylene glycol mono(meth)acrylate or polypropylene glycol mono(meth)acrylate.

Polar or polarizable groups units may be derived from substituted or unsubstituted ammonium monomers of the general formula:

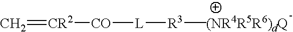

where R², R³, R⁴, R⁵, L and d are as defined above, and where R⁶ is H or alkyl of 1-12 carbon atoms and Q⁻ is an organic or inorganic anion. Preferred examples of such monomers include 2-N,N,N-trimethylammonium ethyl (meth)acrylate, 2-N,N,N-triethylammonium ethyl (meth)acrylate, 3-N,N,N-trimethylammonium propyl (meth)acrylate, N-(2-N′,N′,N′-trimethylammonium)ethyl(meth)acrylamide, N-(dimethyl hydroxyethyl ammonium)propyl(meth)acrylamide, or combinations thereof, where the counterion may include fluoride, chloride, bromide, acetate, propionate, laurate, palmitate, stearate, or combinations thereof. The monomer can also be N,N-dimethyl diallyl ammonium salt of an organic or inorganic counterion.

Ammonium group containing polymers can also be prepared by using as the polar or polarizable group any of the amino group containing monomers described above, and acidifying the resultant polymers with organic or inorganic acid to a pH where the pendant amino groups are substantially protonated. Totally substituted ammonium group containing polymers may be prepared by alkylating the above described amino polymers with alkylating groups, the method being commonly known in the art as the Menschutkin reaction.

Polar or polarizable groups can also be derived from sulfonic acid group containing monomers, such as vinyl sulfonic acid, styrene sulfonic acid, 2-acrylamido-2-methyl propane sulfonic acid, allyloxybenzene sulfonic acid, and the like. Alternatively, polar or polarizable groups may be derived from phosphorous acid or boron acid group-containing monomers. These monomers may be used in the protonated acid form as monomers and the corresponding polymers obtained may be neutralized with an organic or inorganic base to give the salt form of the polymers.

Preferred repeating units of a polar or polarizable group include acrylic acid, itaconic acid, N-isopropylacrylamide, or combinations thereof.

In certain embodiments, the film formers disclosed herein also include a repeating unit that includes a fluoride releasing group. A preferred fluoride releasing group includes tetrafluoroborate anions as disclosed, for example, in U.S. Pat. No. 4,871,786 (Aasen et al.). A preferred repeating unit of a fluoride releasing group includes trimethylammoniumethyl methacrylate.

In certain embodiments, the film formers disclosed herein also include a repeating unit that includes a hydrophobic hydrocarbon group. An exemplary hydrophobic hydrocarbon group is derived from an ethylenically unsaturated preformed hydrocarbon moiety having a weight average molecular weight greater than 160. Preferably the hydrocarbon moiety has a molecular weight of at least 160. Preferably the hydrocarbon moiety has a molecular weight of at most 100,000, and more preferably at most 20,000. The hydrocarbon moiety may be aromatic or non-aromatic in nature, and optionally may contain partially or fully saturated rings. Preferred hydrophobic hydrocarbon moieties are dodecyl and octadecyl acrylates and methacrylates. Other preferred hydrophobic hydrocarbon moieties include macromonomers of the desired molecular weights prepared from polymerizable hydrocarbons, such as ethylene, styrene, alpha-methyl styrene, vinyltoluene, and methyl methacrylate.

In certain embodiments, the film formers disclosed herein also include a repeating unit that includes a hydrophobic fluorine containing group. Exemplary repeating units of hydrophobic fluorine-containing groups include the addition polymerization product of acrylic or methacrylic acid esters of 1,1-dihydroperfluoroalkanols and homologs: CF₃(CF₂)_{x′}CH₂OH and CF₃(CF₂)_{x′}(CH₂)_{y}OH, where x′ is zero to 20 and y is at least 1 up to 10; ω-hydrofluoroalkanols (HCF₂(CF₂)_{x′}(CH₂)_{y}OH), where x′ is 0 to 20 and y is at least 1 up to 10; fluoroalkylsulfonamido alcohols; cyclic fluoroalkyl alcohols; and CF₃(CF₂CF₂O)_{q}(CF₂O)_{x′}(CH₂)_{y}OH, where q is 2 to 20 and greater than x′, x′ is 0 to 20, and y is at least 1 up to 10.

Preferred repeating units of a hydrophobic fluorine-containing group include those derived from 2-(methyl(nonafluorobutyl)sulfonyl)amino)ethyl acrylate, 2-(methyl(nonafluorobutyl)sulfonyl)amino)ethyl methacrylate, or a combination thereof.

In certain embodiments, the film formers disclosed herein also include a repeating unit that includes a graft polysiloxane chain. The graft polysiloxane chain is derived from an ethylenically unsaturated preformed organosiloxane chain. The molecular weight of this unit is generally above 500. Preferred repeating units of a graft polysiloxane chain include a silicone macromer.

Monomers used to provide the graft polysiloxane chain of this invention are terminally functional polymers having a single ethylenically unsaturated functional group (e.g., vinyl, acryloyl, or methacryloyl group) and are sometimes termed macromonomers or “macromers”. Such monomers are known and may be prepared by methods as disclosed, for example, in U.S. Pat. Nos. 3,786,116 (Milkovich et al.) and 3,842,059 (Milkovich et al.). The preparation of polydimethylsiloxane macromonomer and subsequent copolymerization with vinyl monomer have been described in several papers by Y. Yamashita et al., [Polymer J. 14, 913 (1982); ACS Polymer Preprints 25 (1), 245 (1984); Makromol. Chem. 185, 9 (1984)].

In certain embodiments, the film formers disclosed herein also include a repeating unit that includes a modulating group. Exemplary modulating groups are derived from acrylate or methacrylate or other vinyl polymerizable starting monomers and optionally contain functionalities that modulate properties such as glass transition temperature, solubility in the carrier medium, hydrophilic-hydrophobic balance and the like.

Examples of modulating groups include the lower to intermediate methacrylic acid esters of 1 to 12 carbon straight, branched, or cyclic alcohols. Other examples of modulating groups include styrene, vinyl esters, vinyl chloride, vinylidene chloride, acryloyl monomers, and the like.

For certain embodiments, preferred film formers are acrylate-based copolymers and urethane polymers such as the AVALURE series of compounds (e.g., AC-315 and UR-450), and carbomer-based polymers such as the CARBOPOL series of polymers (e.g., 940NF), all available from Noveon, Inc., Cleveland, Ohio.

Film formers can also include caseinates, which are salts and/or complexes of a casein. Casein is a mixture of related phosphoproteins occurring in milk and cheese. Casein is amphoteric and forms salts with both acids and bases. When both the cation and anion of a species (e.g., calcium phosphate) form salts with casein, the product is typically referred to as a complex (e.g., a calcium phosphate complex of casein). Typical caseinates include, for example, salts of monovalent metals (e.g., sodium and potassium), salts of divalent metals (e.g., magnesium, calcium, strontium, nickel, copper, and zinc), salts of trivalent metals (e.g., aluminum), ammonium salts, phosphate salts (e.g., phosphate and fluorophosphate), and combinations thereof. Typical caseinate complexes include, for example, calcium phosphate complexes (available under the trade designation PHOSCAL from NSI Dental Pty. Ltd., Hornsby, Australia), calcium fluorophosphate complexes, calcium fluoride complexes, and combinations thereof. Caseinates are commercially available as dry powders.

### Particulate Sources of Calcium And Phosphorous

As indicated above, particulate sources of calcium and phosphorous include a glass, a glass-ceramic, nanoparticles, nanoclusters, active treated particles, amorphous calcium phosphate, or a combination thereof.

Calcium and phosphorus releasing glasses include calcium and phosphorus in a glass that preferably allows them to be released when placed in the oral environment. Such glasses have been described in the literature as “remineralizing” or, with respect to medical applications, “bioactive.” Such glasses may be melt or sol-gel derived, and may be amorphous or include one or more crystalline phases (i.e., partially crystalline).

Glasses which are sol-gel derived are glass-ceramics.

Remineralizing or bioactive glasses are well known to one of skill in the art, and typical glasses are described, for example, in U.S. Pat. Nos. 6,338,751 (Litkowski et al.) and 6,709,744 (Day et al.), and U.S. Patent Application Publication Nos. 2003/0167967 (Narhi et al.) and 2004/0065228 (Kessler et al.). Exemplary remineralizing or bioactive glasses are available, for example, under the trade designations CERABONE A/W from Nippon Electric Glass Co., Ltd. (Shiga, Japan), BIOVERIT as described by Holand and Vogel in Introduction to Bioceramics, L. L. Hench and J. Wilson, eds., World Scientific Publishing (1993), 45S5 and 45S5F as described by Hench and Andersson in Introduction to Bioceramics, L. L. Hench and J. Wilson, eds., World Scientific Publishing (1993).

In some embodiments, the calcium and phosphorus releasing glass does not include high levels of aluminum oxide (e.g., alumina), which is known to hinder bone mending in medical applications. Such glasses without high levels of aluminum oxide include less than 5%, and sometimes less than 3%, 2%, or even 1% by weight aluminum oxide. In contrast, ionomer glass compositions generally rely on a sufficiently high level of leachable aluminum ions for the ionomeric crosslinking reaction, typically 10-45% by weight Al₂O₃.

In some embodiments, the calcium and phosphorus releasing glass includes 35% to 60% by weight silica, and preferably 40% to 60% by weight silica.

In some embodiments, the calcium and phosphorus releasing glass includes less than 20%, and sometimes less than 15%, 10%, 5%, 3%, or even 1% by weight silica.

In some embodiments, the calcium and phosphorus releasing glass includes at least 15%, and sometimes at least 20%, 25%, 30%, 35%, or even 40% by weight phosphorus pentoxide (P₂O₅). In such embodiments, the calcium and phosphorus releasing glass includes at most 80%, and sometimes at most 75%, 70%, 65%, 60%, 55%, 50%, 45%, or even 40% by weight phosphorus pentoxide (P₂O₅).

In some embodiments, the calcium and phosphorus releasing glass includes less than 20%, and sometimes less than 15%, 12%, 8%, or even 6% by weight phosphorus pentoxide (P₂O₅). In such embodiments, the calcium and phosphorus releasing glass includes at least 1%, and sometimes at least 2%, or even 3% by weight phosphorus pentoxide (P₂O₅).

In some embodiments, the calcium and phosphorus releasing glass includes at least 10%, and sometimes at least 15%, 20%, 25%, or even 30% by weight calcium oxide. In such embodiments, the calcium and phosphorus releasing glass includes at most 70%, and sometimes at most 60%, 50%, 40%, or even 35% by weight calcium oxide.

In some embodiments, the calcium and phosphorus releasing glass optionally includes at most 25%, and sometimes at most 20%, 15%, 10%, or even 5% by weight fluoride.

In some embodiments, the calcium and phosphorus releasing glass optionally includes at most 60%, and sometimes at most 55%, 50%, 45%, 40%, 35%, or even 30% by weight of SrO, MgO, BaO, ZnO, or combinations thereof. In some embodiments, the calcium and phosphorus releasing glass optionally includes at least 0.5%, and sometimes at least 1%, 5%, 10%, 15%, or even 20% by weight of SrO, MgO, BaO, ZnO, or combinations thereof.

In some embodiments, the calcium and phosphorus releasing glass optionally includes at most 40%, and sometimes at most 35%, 30%, 25%, 20%, 15%, 10%, or even 5% by weight rare earth oxide.

In some embodiments, the calcium and phosphorus releasing glass optionally includes at most 45%, and sometimes at most 40%, 30%, 20%, 10%, 8%, 6%, 4%, 3%, or even 2% by weight of Li₂O, Na₂O, K₂O, or combinations thereof. In some embodiments, the calcium and phosphorus releasing glass optionally

includes at most 40%, and sometimes at most 30%, 25%, 20%, 15%, 10%, or even 5% by weight of B₂O₃.

In some embodiments, the calcium and phosphorus releasing glass includes less than 15%, and sometimes less than 10%, 5%, or even 2% by weight of ZrO₂.

In some embodiments, the calcium and phosphorus releasing glass includes 40 to 60% by weight SiO₂, 10 to 35% by weight CaO, 1 to 20% by weight P₂O₅, 0 to 35% by weight Na₂O, and less than 5% by weight Al₂O₃.

In some embodiments, the calcium and phosphorus releasing glass includes 10 to 70% by weight CaO; 20 to 60% by weight P₂O₅; less than 3% by weight Al₂O₃; 0 to 50% by weight of SrO, MgO, BaO, ZnO, or combinations thereof; and less than 10% by weight Li₂O, Na₂O, and K₂O combined.

In some embodiments, the calcium and phosphorus releasing glass includes 10 to 70% by weight CaO; 20 to 50% by weight P₂O₅; less than 3% by weight Al₂O₃; 0 to 50% by weight of SrO, MgO, BaO, ZnO, or combinations thereof; and less than 10% by weight Li₂O, Na₂O, and K₂O combined.

In some embodiments, the calcium and phosphorus releasing glass includes 10 to 50% by weight CaO, at least 15% and less than 50% by weight P₂O₅, less than 3% by weight Al₂O₃, less than 10% by weight Li₂O, Na₂O, and K₂O combined, and 0 to 60% by weight of SrO, MgO, BaO, ZnO, or combinations thereof.

The glass or glass-ceramic may be in a variety of finely divided forms including particles, fibers, or platelets. The preferred average particle size for dental and orthodontic applications is less than 50 micrometers, more preferably less than about 10 micrometers, most preferably less than 3 micrometers. Nanoparticles include glass or glass-ceramic particles with an average particle diameter of less than 0.5 micrometers and preferably less than 0.1 micrometers. Nanoclusters are clusters of nanoparticles, wherein the nanoparticles are associated by relatively weak intermolecular forces that cause the nanoparticles to clump together, even when dispersed in a gel, paste, or hardenable resin, for example, for a dental material. Combinations of different size ranges can also be used.

Calcium and phosphorus releasing glasses can optionally be surface treated (e.g. with silane; acid- or acid-methacrylate monomers, oligomers, or polymers; other polymers, etc.) as described herein below. Such surface treatments can result, for example, in improved bonding of the particles to a matrix. Preferably, the glass is surface treated by methods similar to those described, for example, in U.S. Pat. No. 5,332,429 (Mitra et al.). In brief, the glass can be surface treated by combining the glass with one or more liquids having dissolved, dispersed, or suspended therein, a surface treating agent (e.g., fluoride ion precursors, silanes, titanates, etc). Optionally the one or more liquids include water, and if an aqueous liquid is used, it can be acidic or basic. Once treated, at least a portion of the one or more liquids can be removed from the surface treated glass using any convenient technique (e.g., spray drying, oven drying, gap drying, lyophilizing, and combinations thereof). See, for example, U.S. Pat. No. 5,980,697 (Kolb et al.) for a description of gap drying. In one embodiment, the treated glass can be oven dried, typically at drying temperatures of about 30° to about 100° C., for example, overnight. The surface treated glass can be further heated as desired. The treated and dried glass can then be screened or lightly comminuted to break up agglomerates. The resulting surface treated glass can be incorporated, for example, into a dental paste.

Active treated particles include dental fillers with a treated surface. The treated surface includes phosphorus and a divalent cation selected from the group consisting of Mg, Ca, Sr, Ba, Zn, and combinations thereof. Phosphorus precursors and divalent cation precursors can be used to treat the surface of dental fillers. Phosphorus precursors can be the same as or different than divalent cation precursors. Preferably, the divalent cation precursor includes Mg, Ca, Sr, Ba, Zn, or a combination thereof as divalent cation.

Suitable precursors for phosphorus include, for example, phosphoric acid and salts thereof (e.g., sodium phosphate, potassium phosphate, calcium phosphate, magnesium phosphate, etc.), pyrophosphoric acid and salts thereof (e.g., tetrasodium pyrophosphate, calcium pyrophosphate), monofluorophosphoric acid and salts thereof, hexafluorophosphoric acid and salts thereof, phosphate esters (e.g., triethylphosphate), glycerophosphates (e.g., calcium glycerophosphate, zinc glycerophosphate, magnesium glycerophosphate, strontium glycerophosphate, tin glycerophosphate, zirconium glycerophosphate, and silver glycerophosphate), caseinates (e.g., calcium phosphate complexed caseinates), phosphorous oxides (e.g., P₂O₅), phosphorus oxyhalides (e.g., POCl₃), and combinations thereof.

Suitable precursors for divalent cations include organic and inorganic salts of the cation with an anion, and basic or oxy salts thereof. Exemplary anions include, for example, nitrate, halide (e.g., chloride, fluoride, etc.), hydroxide, alkoxide, caseinate, carboxylate (e.g., formate, acetate, formoacetate), and combinations thereof.

In addition, precursors for other cations (e.g., trivalent cations) and/or anions (e.g., fluoride ion) may optionally be used to surface treat the dental fillers. For example, suitable precursors for trivalent cations (e.g., aluminum, lanthanum, or combinations thereof) include, for organic and inorganic salts of the cation with an anion, and basic or oxy salts thereof. Exemplary anions include, for example, nitrate, halide (e.g., chloride, fluoride, etc.), hydroxide, alkoxide, caseinate, carboxylate (e.g., formate, acetate, formoacetate), and combinations thereof. Suitable precursors for fluoride ion include, for example, ammonium fluoride, ammonium hydrogen difluoride, hexafluorosilicic acid and salts thereof, monofluorophosphoric acid and salts thereof, hexafluorophosphoric acid and salts thereof, and combinations thereof.

The active treated particles can be made by treating the surface of a dental filler using methods similar to those described, for example, in U.S. Pat. No. 5,332,429 (Mitra et al.). In brief, a dental filler can be surface treated by combining the filler with one or more liquids having dissolved, dispersed, or suspended therein, a phosphorus precursor and a divalent cation precursor as described above. The one or more liquids or additional liquids may optionally include additional surface treating agents (e.g., fluoride ion precursors, silanes, titanates, etc). Optionally the one or more liquids include water, and if an aqueous liquid is used, it can be acidic or basic. Once treated, at least a portion of the one or more liquids can be removed from the surface treated dental filler using any convenient technique (e.g., spray drying, oven drying, gap drying, lyophilizing, and combinations thereof). See, for example, U.S. Pat. No. 5,980,697 (Kolb et al.) for a description of gap drying. In one embodiment, the treated fillers can be oven dried, typically at drying temperatures of about 30° to about 100° C., for example, overnight. The surface treated filler can be further heated as desired. The treated and dried dental filler can then be screened or lightly comminuted to break up agglomerates. The resulting active particles can be combined with a substantially anhydrous liquid to form a composition of the present invention.

Dental fillers suitable for surface treatment can be selected from one or more of a wide variety of materials suitable for incorporation in compositions used for dental applications, such as fillers currently used in dental restorative compositions, and the like. Preferably the dental filler includes porous particles and/or porous agglomerates of particles. Preferred dental fillers include nanoparticles and/or agglomerates of nanoparticles. Preferred classes of fillers include metal oxides, metal fluorides, metal oxyfluorides, and combinations thereof, wherein the metal can be a heavy or non-heavy metal.

For certain embodiments, the dental filler is preferably an oxide, a fluoride, or an oxyfluoride of an element selected from the group consisting of Groups 2-5 elements, Groups 12-15 elements, Lanthanide elements, and combinations thereof. More preferably, the element is selected from the group consisting of Ca, Sr, Ba, Y, La, Ce, Pr, Nd, Pm, Sm Eu, Gd, Tb, Dy, Ho, Er, Tm Yb, Lu, Ti, Zr, Ta, Zn B, Al, Si, Sn, P, and combinations thereof. The dental filler can be a glass, an amorphous material, or a crystalline material. Optionally, the dental filler can include a source of fluoride ions. Such dental fillers include, for example, fluoroaluminosilicate glasses.

The filler is preferably finely divided. The filler can have a unimodal or polymodal (e.g., bimodal) particle size distribution. Preferably, the maximum particle size (the largest dimension of a particle, typically, the diameter) of the filler is less than 20 micrometers, more preferably less than 10 micrometers, and most preferably less than 5 micrometers. Preferably, the average particle size of the filler is less than 2 micrometers, more preferably less than 0.1 micrometers, and most preferably less than 0.075 micrometer.

The filler can be an inorganic material. It can also be a crosslinked organic material that is insoluble in the resin system, and is optionally filled with inorganic filler. The filler should in any event be nontoxic and suitable for use in the mouth. The filler can be radiopaque or radiolucent. The filler typically is substantially insoluble in water.

Examples of suitable inorganic fillers are naturally occurring or synthetic materials including, but not limited to: quartz; nitrides (e.g., silicon nitride); glasses derived from, for example, Zr, Sr, Ce, Sb, Sn, Ba, Zn, and Al; feldspar; borosilicate glass; kaolin; talc; titania; low Mohs hardness fillers such as those described in U.S. Pat. No. 4,695,251 (Randklev); and submicron silica particles (e.g., pyrogenic silicas such as those available under the trade designations AEROSIL, including “OX 50,” “130,” “150” and “200” silicas from Degussa Corp., Akron, Ohio and CAB-O-SIL M5 silica from Cabot Corp., Tuscola, Ill.). Examples of suitable organic filler particles include filled or unfilled pulverized polycarbonates, polyepoxides, and the like.

Preferred non-acid-reactive filler particles are quartz, submicron silica, and non-vitreous microparticles of the type described in U.S. Pat. No. 4,503,169 (Randklev). Mixtures of these non-acid-reactive fillers are also contemplated, as well as combination fillers made from organic and inorganic materials. Silane-treated zirconia-silica (Zr—Si) filler is especially preferred in certain embodiments.

The filler can also be an acid-reactive filler. Suitable acid-reactive fillers include metal oxides, glasses, and metal salts. Typical metal oxides include barium oxide, calcium oxide, magnesium oxide, and zinc oxide. Typical glasses include borate glasses, phosphate glasses, and fluoroaluminosilicate (“FAS”) glasses. FAS glasses are particularly preferred. The FAS glass typically contains sufficient elutable cations so that a hardened composition will form when the glass is mixed with appropriate components of the hardenable composition. The glass also typically contains sufficient elutable fluoride ions so that the hardened composition will have cariostatic properties. The glass can be made from a melt containing fluoride, alumina, and other glass-forming ingredients using techniques familiar to those skilled in the FAS glassmaking art. The FAS glass typically is in the form of particles that are sufficiently finely divided so that they can conveniently be mixed with the other components and will perform well when the resulting mixture is used in the mouth.

Generally, the average particle size (typically, diameter) for the FAS glass is no greater than about 12 micrometers, typically no greater than 10 micrometers, and more typically no greater than 5 micrometers as measured using, for example, a sedimentation analyzer. Suitable FAS glasses will be familiar to those skilled in the art, and are available from a wide variety of commercial sources, and many are found in currently available glass ionomer cements such as those commercially available under the trade designations VITREMER, VITREBOND, RELY X LUTING CEMENT, RELY X LUTING PLUS CEMENT, PHOTAC-FIL QUICK, KETAC-MOLAR, and KETAC-FIL PLUS (3M ESPE Dental Products, St. Paul, Minn.), FUJI II LC and FUJI IX (G-C Dental Industrial Corp., Tokyo, Japan) and CHEMFIL Superior (Dentsply International, York, Pa.). Mixtures of fillers can be used if desired.

Other suitable fillers are disclosed, for example, in U.S. Pat. Nos. 6,306,926 (Bretscher et al.), 6,387,981 (Zhang et al.), 6,572,693 (Wu et al.), and 6,730,156 (Windisch et al.), as well as International Publication Nos. WO 01/30307 (Zhang et al.) and WO 03/063804 (Wu et al.). Filler components described in these references include nanosized silica particles, nanosized metal oxide particles, and combinations thereof. Nanofillers are also described in U.S. Pat. Nos. 7,090,721 (Craig et al.) and 7,156,911 (Kangas et al.); and U.S. Publication No. 2005/0256223.

The surface treated dental filler preferably includes at least 0.01%, more preferably at least 0.05%, and most preferably at least 0.1% by weight phosphorus, based on the total dry weight of the dental filler (i.e., excluding the liquid used in the treatment). The surface treated dental filler preferably includes at most 50%, more preferably at most 30%, and most preferably at most 20% by weight phosphorus, based on the total dry weight of the dental filler (i.e., excluding any liquid used in a treatment).

The surface treated dental filler preferably includes at least 0.01%, more preferably at least 0.05%, and most preferably at least 0.1% by weight divalent cation, based on the total dry weight of the dental filler (i.e., excluding the liquid used in the treatment). The surface treated dental filler preferably includes at most 50%, more preferably at most 30%, and most preferably at most 20% by weight divalent cation, based on the total dry weight of the dental filler (i.e., excluding any liquid used in a treatment).

### Fillers

Compositions as described herein may optionally include fillers, which optionally may be surface treated in a manner similar to the treatment of the calcium and phosphorus glasses as described herein. Suitable fillers include those described above.

For certain embodiments of the present invention that optionally include a dental filler (e.g., dental adhesive compositions), the compositions preferably include at least 1% by weight, more preferably at least 2% by weight, and most preferably at least 5% by weight dental filler, based on the total weight of the composition. For such embodiments, compositions of the present invention preferably include at most 40% by weight, more preferably at most 20% by weight, and most preferably at most 15% by weight dental filler, based on the total weight of the composition.

For other embodiments that optionally include a dental filler (e.g., wherein the composition is a dental restorative or an orthodontic adhesive), compositions of the present invention preferably include at least 40% by weight, more preferably at least 45% by weight, and most preferably at least 50% by weight dental filler, based on the total weight of the composition. For such embodiments, compositions of the present invention preferably include at most 90% by weight, more preferably at most 80% by weight, even more preferably at most 70% by weight, and most preferably at most 50% by weight dental filler, based on the total weight of the composition.

Optionally, the dental filler can include a treated surface that further includes a silane (e.g., as described, for example, in U.S. Pat. No. 5,332,429 (Mitra et al.)), an antibacterial agent (e.g., chlorhexidine; quaternary ammonium salts; metal containing compounds such as Ag, Sn, or Zn containing compounds; and combinations thereof), and/or a source of fluoride ions (e.g., fluoride salts, fluoride containing glasses, fluoride containing compounds, and combinations thereof).

### Optional Additives

If desired, the compositions of the invention can further include additives such as fillers, dental abrasives, abrasive polishing material, indicators, anticalculus agents, tartar control agents, antiplaque agents, antigingivitis agents, colorants (including dyes and pigments), fluorescence imparting agents, opalescence imparting agents, opacifiers, inhibitors, accelerators, rheology modifiers, wetting agents, acidifying agents, tartaric acid, basifying agents, chelating agents, buffering agents, diluents, stabilizers, humectants, foaming agents, for example, sodium lauryl sulfate, emulsifiers, surfactants, nutrients, flavorants, sweeteners, agents to alleviate halitosis, and other similar ingredients. Additionally, medicaments or other therapeutic substances can be optionally added to the dental compositions. Examples include, but are not limited to, enzymes, breath fresheners, anesthetics, anticaries agents, antigingivitis agents, antimicrobial agents (e.g., triclosan, fatty acid monoesters, chlorhexidine gluconate, benzalkonium chloride, glutaraldehyde, quaternary ammonium salts, and guanidines), clotting agents, acid neutralizers, chemotherapeutic agents, immune response modifiers, thixotropes, anti-inflammatory agents, and the like, of the type often used in dental compositions. Combinations of any of the above additives may also be employed.

For certain embodiments, including any one of the compositions described herein, the composition further comprises an additional component selected from the group consisting of fillers, dental abrasives, rheology modifiers, anticaries agents, antigingivitis agents, flavors, colorants, diluents, antimicrobial agents, pH control agents, stabilizers, and combinations thereof. For certain of these embodiments, the additional component is a dental abrasive. Suitable dental abrasives include silicas (including gels and precipitates); aluminas; phosphates (including orthophosphates, polymetaphosphates, and pyrophosphates); and mixtures thereof. Specific examples include dicalcium orthophosphate dihydrate, calcium pyrophosphate, tricalcium phosphate, calcium polymetaphosphate, insoluble sodium polymetaphosphate, hydrated alumina, beta calcium pyrophosphate, calcium carbonate, and resinous abrasive materials. Preferred silicas include the silica xerogels (available under the trade name “Syloid” from W. R. Grace & Company), and precipitated silica materials such as those available under the trade name “Zeodent” from J. M. Huber Corporation.

### Preparation of the Compositions

Compositions disclosed herein can be prepared by adding an ion source compound, for example, a divalent metal cation source, such as a salt of a divalent metal cation, to a substantially anhydrous liquid with mixing until the ion source compound is dissolved in the substantially anhydrous liquid. For compositions which include a metal cation and an anion, such as a phosphate anion, separate sources of the cation and the anion can be added sequentially or at the same time to the substantially anhydrous liquid with mixing to dissolve the cation and the anion in the substantially anhydrous liquid and form the composition. Mixing can conveniently be carried out at room temperature, for example, at 25° C.

Alternatively, each source can be added to a separate quantity of the substantially anhydrous liquid with mixing to separately dissolve the cation and anion in the substantially anhydrous liquid. The resulting solutions are then combined to provide a composition with both the cation and anion dissolved in the substantially anhydrous liquid. As described above, this has now been found to be an effective method where the anion is a glycerophosphate or the like.

Other components can be added before, during, or after dissolving the divalent metal cations and/or anions as described above. For example, matrix forming components, other metal cations, anticaries agents, and bleaching agents can be dissolved in the substantially anhydrous liquid before, during, or after the divalent metal cations and/or anions are dissolved therein. Other components, such as fillers, which do not dissolve, are preferably added after the divalent metal cations and/or anions are dissolved in the substantially anhydrous liquid. This allows verification that the desired solution is formed without interference by these other components.

For certain embodiments, each ion source (or ion source compound) is present in a concentration of at least 0.002% by weight of the composition. In some embodiments, the level of each ion source is at least about 0.005%, 0.01%, 0.02%, 0.05%, 0.07%, 0.1%, 0.2%, 0.4%, 0.6%, 0.8%, or even at least about 1%. When more than one ion source is present, the concentration of each ion source may be the same or different. The levels and ratios of ion sources are selected based on several considerations including providing at least one target benefit, immediate and long-term stability, and overall optimization of all key attributes of the composition. A stable composition is one that lacks precipitation of the ion sources, adverse reaction with other ingredients, and adverse changes in key attributes during storage. Compositions incorporating dispersed particles (e.g. abrasives, fillers, etc.) may, in certain embodiments, have reduced stable levels of ion sources compared to the corresponding unfilled composition. For certain embodiments, the maximum amount of each ion source is limited by solubility in the liquid system. For certain embodiments, the maximum amount of each ion source is not more than 30, 20, 10, 5, 2, or 1 weight percent of the composition.

Particulate sources, such as particulate sources of calcium, phosphorous, and fluoride, are dispersed in the composition and, although not limited by solubility in the liquid system, may be present in the amounts described above for the ion source.

For certain embodiments, compositions which include anticaries and/or bleaching agents have these materials present independently at a concentration of at least 0.01, 0.02, 0.05, 0.1, 0.2, 0.5, or 1 percent by weight of the composition. For certain of these embodiments, the anticaries and/or bleaching agent is present independently at a concentration of not more than 30, 20, 15 or 10 percent by weight of the composition.

For certain embodiments, compositions which include a matrix forming component have this component present at a concentration of at least 1, 2, or 5 percent by weight of the composition. For certain of these embodiments, the matrix forming component is present at a concentration of not more than 95, 75, 50, or 30 percent by weight of the composition.

### Methods of Use

When the composition is a hardenable composition (e.g., includes a polymerizable resin), the composition may contain a photoinitiator system and be hardened by photoinitiation, or may contain a thermal initiator system and be hardened by chemical polymerization such as a redox cure mechanism. Alternatively, the hardenable composition may contain an initiator system such that the composition can be both a photopolymerizable and a chemically polymerizable composition.

As indicated above, certain compositions of the present invention can be supplied as a one-part system or as a multi-part system, e.g., two-part liquid/liquid, powder/liquid, paste/liquid, and paste/paste systems. Other forms employing multi-part combinations (i.e., combinations of two or more parts), each of which is in the form of a powder, liquid, gel, or paste are also possible. In a redox multi-part system, one part typically contains the oxidizing agent and another part typically contains the reducing agent. The components of such compositions can be included in a kit, where the contents of the composition are packaged to allow for storage of the components until they are needed. The components of compositions of the present invention can be mixed and clinically applied using conventional techniques.

Exemplary methods of using compositions of the present invention are described in the Examples. In some embodiments, the present invention provides a method of treating a tooth structure, comprising contacting the tooth structure with any one of the above compositions. For certain of these embodiments, the treatment provides a benefit selected from the group consisting of xerostomia relief, enamel conditioning, lesion reduction, desensitization, halitosis relief, and a combination thereof. Compositions described herein can help replenish the supply of calcium and phosphate ions when these ions are depleted by a xerostomia condition.

In some embodiments, there is provided a method of remineralizing a tooth structure, comprising placing any one of the above compositions in an oral environment.

In some embodiments, there is provided a method of reducing the sensitivity of a tooth structure, comprising placing any one of the above the compositions in an oral environment.

In some embodiments, there is provided a method of protecting a tooth structure, comprising placing any one of the above compositions in an oral environment.

In some embodiments, there is provided a method of delivering a plurality of ions to an oral environment comprising placing any one of the above compositions in the oral environment. For certain of these embodiments, the plurality of ions comprises an element selected from the group consisting of calcium, phosphorous, and a combination thereof.

In some embodiments, there is provided a method of preparing a dental article comprising hardening any one of the above composition which includes a polymerizable resin to fabricate a dental article selected from the group consisting of crowns, fillings, mill blanks, orthodontic devices, and prostheses.

Objects and advantages of this invention are further illustrated by the following examples, but the particular materials and amounts thereof recited in these examples, as well as other conditions and details, should not be construed to unduly limit this invention. Unless otherwise indicated, all parts and percentages are on a weight basis, all water is deionized water, and all molecular weights are weight average molecular weight.

### EXAMPLES

Unless otherwise indicated, all percent values are percent by weight.

### Test Methods

### Ion Release From Compositions

Ion release from gel and paste compositions was measured as follows. Four grams of paste or gel was blended with 12 ml of deionized water with vigorous mixing for 1 minute. The resulting mixture was centrifuged immediately for 10 minutes. The resulting supernate was then recovered and analyzed. Calcium and fluoride ion concentrations were measured with ion-selective electrodes (Orion Calcium electrode 9720BN; Orion Fluoride Combination electrode, model 96-09; both from Thermo Electron Corporation, Beverly, Mass.) according to the manufacturer's instructions. This sample preparation was adapted from Test 3A, “Measuring the One Minute Fluoride Release Rate of NaF & SnF₂ Dentifrices,” in Fluoride-Containing Dentifrices, published by the American Dental Association council on Scientific Affairs, 2005. This is a guidance document for the ADA Acceptance Program. This test is intended to measure the effective ion release from an oral paste or gel (e.g., dentifrice, prophy, gel) during a typical one minute application period.

### Ion Release From Coatings

Ion release from coatings made using compositions described herein was measured as follows. A thin layer of the coating material was brushed onto the frosted surface of a glass slide, using a fiber-tipped brush (available from 3M ESPE). The resulting coating was allowed to air-dry for 30 minutes to provide a coated slide, which was placed into 30 ml of deionized water at 37° C. in a jar. The jar was sealed and stored at 37° C. The water was replaced at time intervals of 20 minutes, 2 hours, and 24 hours. The calcium ion concentration in each leachate solution (the water that was replaced at each time interval) was measured, using a calcium selective electrode as described above. The calcium ion concentration was reported as micrograms calcium/gram solution.

### Ion Recharge of Coatings

Ion recharge of coatings, using paste or gel compositions described herein to recharge the coatings, was measured as follows. A thin layer of the coating material was brushed onto the frosted surface of a glass slide, using a fiber-tipped brush (available from 3M ESPE). The resulting coating was allowed to air-dry for 30 minutes to provide a coated slide, which was placed into 30 ml of deionized water at 37° C. in a jar. The jar was sealed and stored at 37° C. for 24 hours. The calcium ion concentration of the water was measured initially (at time=0) and at 24 hours to establish the baseline concentration. The coated slide was then removed from the water and treated for 2 minutes with a slurry made from the paste or gel composition and water in a ratio of 1:1. The treatment was carried out by gently swabbing the coating with the slurry using a pre-moistened cotton swab. The coated slide was then sonicated in deionized water for 1 minute, rinsed thoroughly with deionized water, and placed in 30 ml of fresh deionized water for an additional 24 hours at 37° C. The calcium ion concentration of the water was then measured. The calcium ion concentrations were measured using a calcium-selective electrode as described above, and the concentrations were reported as parts per million (ppm) calcium.

### Dentin Tubule Occlusion

In some embodiments, remineralizing compositions may be used to occlude open dentin tubules, which can be the cause of dentin and root sensitivity. The ability of a composition to occlude dentin tubules was determined as follows. A slab of bovine dentin, cut with a slow-speed diamond wafer saw, was etched for 1 minute with phosphoric acid etchant (available from 3M ESPE as SCOTCHBOND ETCH OR ATZGEL/ETCH GEL MINITIP), sonicated for 1 minute in deionized water, and rinsed thoroughly in deionized water. The resulting exposed dentin slab was treated for 2 minutes as described in the following Examples. The slab was then sonicated in deionized water for 1 minute, rinsed thoroughly with deionized water, and dried. A scanning electron micrograph (SEM) was taken of the surface of the resulting slab. SEM's of untreated dentin were used as negative controls, which showed open tubules with no deposition or occlusion. Examples of negative control SEM's are shown in FIGS. 4 and 7.

Abbreviations, Descriptions, and Sources of Materials

| | Description and Source of Material (Unless otherwise |
|---|---|
| Abbreviation | indicated, available from Sigma-Aldrich, St. Louis, MO.) |
| AA | acrylic acid |
| DMAEMA | dimethylaminoethyl methacylate |
| IBMA | isobutyl methacrylate |
| BisGMA | 2,2-bis[4-(2-hydroxy-3- |
| | methacryloyloxypropoxy)phenyl]propane CAS No. 1565-94-2 |
| PEGDMA | polyethyleneglycol dimethacrylate (Sartomer 603; MW about 570; |
| | Sartomer, Exton, PA) |
| TEGDMA | triethyleneglycol dimethacrylate |
| HEMA | 2-hydroxyethyl methacrylate |
| BisGMA/HEMA | clear solution of 50% bisGMA and 50% HEMA |
| VBP | polymer made by reacting PAA:ITA copolymer with |
| | sufficient IEM to convert 16 mole percent of the acid groups |
| | of the copolymer to pendent methacrylate groups, according to the |
| | dry polymer preparation of Example 11 of U.S. Pat. |
| | No. 5,130,347 (Mitra). |
| PAA:ITA | copolymer made from a 4:1 mole ratio of acrylic |
| | acid:itaconic acid, prepared according to Example 3 of |
| | U.S. Pat. No. 5,130,347, MW (average) = 106,000; |
| | polydispersity ρ = 4.64. |
| IEM | 2-isocyanatoethyl methacrylate |
| CPQ | camphorquinone |
| TINUVIN P | 2-(2-hydroxy-5-methylphenyl)benzotriazole (available from |
| | Ciba-Geigy Corp, Hawthorne, NY) |
| BHT | 2,6-di-tert-butyl-4-methylphenol |
| EDMAB | ethyl 4-(N,N-dimethylamino)benzoate |
| DPIHFP | diphenyl iodonium hexafluorophosphate (Johnson Matthey, |
| (DPIPF₆) | Alpha Aesar Division, Ward Hill, NJ) |
| DPISbF₆ | diphenyl iodonium hexafluoroantimonate |
| Vitrebond | powder component of VITREBOND Light Cure Glass |
| Powder | Ionomer Liner/Base (3M Company, St. Paul, MN) |
| Vitremer Resin | liquid component of VITREMER Core/Restorative (3M |
| | Company, St. Paul, MN) |
| IADMA | 25% IBMA/AA/DMAEMA (60/20/20) copolymer in ethanol |
| V20H Resin | clear solution of 20% VBP and 80% HEMA |
| GDMA | glycerol dimethacrylate (Rohm Tech, Inc., Malden, MA) |
| CDMA | citric acid dimethacrylate |
| CDMA/GDMA | a mixture of GDMA and CDMA (50/50) (see Preparatory |
| | Example 2 of U.S. Pat. No. 5,922,786 (Mitra et al.) |
| CPVH Resin | 15% VBP, 35% HEMA, 50% CP BLEND, 0.3% CPQ, 1% |
| | DIIPF₆, 0.05% BHT |
| 3CPVH Resin | 15% VBP, 35% HEMA, 50% 3:1 CDMA:PEGDMA |
| OMNI Gel | a solution of 0.4 weight percent SnF₂ in glycerol, along with flavor, |
| | dye, and thickener (available from Omni Oral |
| | Pharmaceuticals, a 3M ESPE company). |
| CP Blend | a 1:1 blend of citric acid dimethacrylate and polyethylene |
| | glycol (MW = 400) dimethacrylate |
| PM2 | KAYAMER PM-2; bis(methacryloxyethyl) phosphate |
| | (Nippon Kiyaku, Japan) |
| BMP Resin | light curable resin containing 32.00% bisEMA6, 32.00% |
| | TEGDMA, 33.15% PM2, 0.3% CPQ, 2.4% EDMAB, and |
| | 0.15% BHT |
| P10V2 Resin | 18% HEMA, 31% BT Blend, 10.34% PM2, 2.00% V20H, |
| | and 35.00% TEGDMA |
| BT Blend | 9:1 blend of bisGMA and TEGDMA |
| BisEMA6 | ethoxylated (6 mole ethylene oxide) Bisphenol A |
| | dimethacrylate (available from Sartomer, Exton, PA) |
| pNVP | poly(N-vinylpyrrolidone), MW (average) of about 58,000 |
| | (available as PLASDONE K-29/32 from International |
| | Specialty Products, Wayne, N.J.) |
| CP Resin | a resin containing 66.06% CDMA/GDMA, 27.63% GDMA, |
| | 4% pNVP, 0.65% TINUVIN P, 0.3% CPQ, 1.25% |
| | EDMAB, and 0.1% BHT |
| BTLC Resin | photocurable resin made by mixing and dissolving: |
| | 0.01 parts by weight (pbw) EDMAB, 0.0017 pbw CPQ, 0.01 |
| | pbw TINUVIN P, 0.006 pbw DPIPF₆, 0.4862 pbw bisGMA, |
| | and 0.4862 pbw TEGDMA |
| AC-315 | AVALURE acrylate-based copolymer (Noveon, Inc., |
| | Cleveland, OH) |
| CARBOPOL | carbomer-based polymer (Noveon, Inc., Cleveland, OH) |
| 974P | |
| pTHF | polytetrahydrofuran, number average MW of 250 |
| UVR-6105 | epoxy resin available from Union Carbide Co. (Danbury, CT) |
| PEG300 | poly(ethylene glycol), number average MW of 300 |
| PG | propylene glycol |
| DCPA | dicalcium phosphate, anhydrous, CaHPO₄ (Alfa Aesar, Ward |
| | Hill, MA) |
| MCPA | monocalcium phosphate, anhydrous, Ca(H₂PO₄)₂ (Sigma- |
| | Aldrich) |
| UHP | urea hydrogen peroxide, CH₄N₂O*H₂O₂ |
| PROSPECT MI | a paste containing RECALDENT, a casein-derived source of |
| | calcium and phosphate (available from GC America) |
| SOOTHERX | a paste containing NOVAMIN glass, a sodium calcium |
| | phosphosilicate glass (available from Omni Oral |
| | Pharmaceuticals, a 3M ESPE company) |
| ADPER | liquid A component of ADPER PROMPT Self-Etch Adhesive |
| PROMPT A | (available from 3M ESPE) |
| ADPER | liquid B component of ADPER PROMPT Self-Etch Adhesive |
| PROMPT B | (available from 3M ESPE) |
| POSTPROPHY | an aqueous acidulated phosphate fluoride solution (available |
| Treatment, | as POSTPROPHY Post Prophylaxis Remineralization |
| Solution A | Treatment, Solution A from Omni Preventive Care, a 3M ESPE |
| | company) |

### Preparation of Starting Solutions

Ion source compounds, including calcium and phosphorous sources were added to the indicated liquids and mixed in vials, using a twin shell dry blender (available from Paterson-Kelley Company, East Stroudsburg, Pa.), to provide clear starting solutions. The following Table 1 shows the composition of each of these starting solutions.

TABLE 1
Starting Solutions 1C-28C, 1F-3F, 1P-30P, 1MI-27MI

| | | | Ion Source |
|---|---|---|---|
| Starting | Substantially | | Concentration |
| Solution | Anhydrous Liquid | Ion Source | (Weight Percent¹) |
| 1C | Ethanol | Ca(NO₃)₂•4H₂O | 30 |
| 2C | Ethanol | CaCl₂ | 20.4 |
| 3C | Glycerol | Ca(NO₃)₂•4H₂O | 10 |
| 4C | Glycerol | Ca(NO₃)₂•4H₂O | 11.4 |
| 5C | Glycerol | Ca(NO₃)₂•4H₂O | 20 |
| 6C | Glycerol | Ca(Na)₂EDTA² | 2.0 |
| 7C | Glycerol | CaCl₂•2H₂O | 5.0 |
| 8C | Glycerol | CaCl₂•2H₂O | 5.6 |
| 9C | Glycerol | CaO₂(O)POCH₂CH₂N⁺(CH₃)₃Cl⁻ | 1.0 |
| 10C | PEG300 | Ca(NO₃)₂•4H₂O | 9.8 |
| 11C | PG | Ca(NO₃)₂•4H₂O | 10.4 |
| 12C | 1-Methoxy-2- | Ca(NO₃)₂•4H₂O | 42.9 |
| | propanol | | |
| 13C | CPVH Resin | Ca(NO₃)₂•4H₂O | 4.0 |
| 14C | CPVH Resin | Ca(NO₃)₂•4H₂O | 5.0 |
| 15C | F2000 Resin | Ca(NO₃)₂•4H₂O | 4.0 |
| 16C | IADMA Resin | Ca(NO₃)₂•4H₂O | 10 |
| 17C | HEMA | Ca(NO₃)₂•4H₂O | 4.0 |
| 18C | P10V2 Resin | Ca(NO₃)₂•4H₂O | 4.0 |
| 19C | bisGMA/HEMA | Ca(NO₃)₂•4H₂O | 4.0 |
| 20C | BMP Resin | Ca(NO₃)₂•4H₂O | 4.0 |
| 21C | V20H Resin | Ca(NO₃)₂•4H₂O | 4.0 |
| 22C | 30:70 | Ca(NO₃)₂•4H₂O | 10 |
| | VBP:HEMA | | |
| | Resin | | |
| 23C | ADPER | Ca(NO₃)₂•4H₂O | 10 |
| | PROMPT A | | |
| 24C | ADPER | Ca(NO₃)₂•4H₂O | 10 |
| | PROMPT B | | |
| 25C | OMNI Gel | Ca(NO₃)₂•4H₂O | 1.7 |
| 26C | PM2 | CaCl₂ | 4.0 |
| 27C | BMP RESIN | Ca(O(O)CCH₃)₂ hydrate | 4.2 |
| 28C | P10V2 Resin | Ca(O(O)CCH₃)₂ hydrate | 4.0 |
| 1F | Glycerol | SnF₂ | 0.4 |
| 2F | Glycerol | SnF₂ | 0.75 |
| 3F | Glycerol | SnF₂ | 1.0 |
| 4F | Glycerol | SnF₂ | 1.64 |
| 1P | Ethanol | P₂O₅ | 21 |
| 2P | Ethanol | NaPF₆ | 2.0 |
| 3P | Glycerol | (NaO)₂(O)POCH(CH₂OH)₂•xH₂O | 10 |
| 4P | Glycerol | (NaO)₂(O)POCH(CH₂OH)₂•xH₂O | 2.9 |
| 5P | Glycerol | Na₂FPO₃ | 1.0 |
| 6P | Glycerol | NH₄PF₆ | 6.0 |
| 7P | Glycerol | NH₄PF₆ | 6.3 |
| 8P | Glycerol | NH₄PF₆ | 15 |
| 9P | Glycerol | NaPF₆ | 2.0 |
| 10P | Glycerol | NaH₂PO₄•2H₂O | 4.0 |
| 11P | Glycerol | NaH₂PO₄•2H₂O | 4.1 |
| 12P | Glycerol | Na₂HPO₄ | 3.1 |
| 13P | Glycerol | KH₂PO₄ | 4.0 |
| 14P | Glycerol | K₂HPO₄ | 4.7 |
| 15P | Glycerol | K₂HPO₄•3H₂O | 4.0 |
| 16P | Glycerol | NaH₂PO₂•H₂O | 2.0 |
| 17P | Glycerol | Creatinine phosphate | 1.0 |
| | | disodium | |
| 18P | PG | NH₄PF₆ | 2.9 |
| 19P | PG | NaPF₆ | 3.1 |
| 20P | PG | (NaO)₂(O)POCH(CH₂OH)₂•xH₂O | 2.9 |
| 21P | PEG300 | NaPF₆ | 2.0 |
| 22P | PEG300 | NaPF₆ | 3.2 |
| 23P | CPVH Resin | NaPF₆ | 4.0 |
| 24P | CP Resin | NaPF₆ | 4.0 |
| 25P | IADMA | NaPF₆ | 2.0 |
| 26P | IADMA | P₂O₅ | 5.0 |
| 27P | HEMA | NaPF₆ | 4.0 |
| 28P | bisGMA/HEMA | NaPF₆ | 4.0 |
| 29P | ADPER | NaPF₆ | 6.0 |
| | PROMPT A | | |
| 30P | ADPER | NaPF₆ | 10 |
| | PROMPT B | | |
| 31P | Glycerol | H₄P₂O₇ | 4 |
| 32P | Ethanol | H₄P₂O₇ | 20 |
| 1MI | Ethanol | Zn(NO₃)₂•6H₂O | 10.0 |
| 2MI | Ethanol | Al(NO₃)₃ hydrate | 4.0 |
| 3MI | Glycerol | Zn(NO₃)₂•6H₂O | 4.7 |
| 4MI | Glycerol | SrNO₃ | 2.0 |
| 5MI | Glycerol | Mg(NO₃)₂•2H₂O | 10.0 |
| 6MI | Glycerol | La(NO₃)₃ hydrate | 5.0 |
| 7MI | Glycerol | Al(NO₃)₃ hydrate | 5.0 |
| 8MI | PEG300 | Zn(NO₃)₂•6H₂O | 4.8 |
| 9MI | PG | Zn(NO₃)₂•6H₂O | 6.3 |
| 10MI | IADMA Resin | Zn(NO₃)₂•6H₂O | 5.5 |
| 11MI | IADMA Resin | Zn(NO₃)₂•6H₂O | 15.4 |
| 12MI | IADMA Resin | La(NO₃)₃ hydrate | 4.0 |
| 13MI | IADMA Resin | La(NO₃)₃ hydrate | 5.0 |
| 14MI | IADMA Resin | Al(NO₃)₃ hydrate | 4.0 |
| 15MI | IADMA Resin | Gd(NO₃)₃ hydrate | 4.0 |
| 16MI | IADMA Resin | Ce(NO₃)₃ hydrate | 4.0 |
| 17MI | P10V2 Resin | Zn(NO₃)₂•6H₂O | 4.0 |
| 18MI | P10V2 Resin | Yb(NO₃)₃ | 4.0 |
| 19MI | BMP Resin | Zn(NO₃)₂•6H₂O | 2.0 |
| 20MI | V20H Resin | Zn(NO₃)₂•6H₂O | 4.0 |
| 21MI | V20H Resin | Yb(NO₃)₃ | 4.0 |
| 22MI | CPHV Resin | Zn(NO₃)₂•6H₂O | 3.3 |
| 23MI | HEMA | La(NO₃)₃ hydrate | 4.0 |
| 24MI | HEMA | Al(NO₃)₃ hydrate | 4.0 |
| 25MI | HEMA | Gd(NO₃)₃ hydrate | 4.0 |
| 26MI | HEMA | Ce(NO₃)₃ hydrate | 4.0 |
| 27MI | BTLC Resin | Gd(NO₃)₃ hydrate | 2.2 |

¹Percent by weight based upon the weight of the solution.
²EDTA = [—O(O)CCH₂]₂NCH₂CH₂N[CH₂C(O)O—]₂

Matrix forming components (polymerizable resins or film formers) were added to the indicated liquid and mixed to form a clear solution. The following Table 2 shows the composition of each of these starting solutions.

TABLE 2
Starting Solutions 1MFC-4MFC

| | | Matrix Forming | MFC Concentration |
|---|---|---|---|
| Starting Solution | Liquid | Component (MFC) | Weight Percent¹ |
| 1MFC | Ethanol | IADMA | 25 |
| 2MFC | Ethanol | AC315 | 25 |
| 3MFC | Ethanol | AC315 | 30 |
| 4MFC | Ethanol | AC315 | 28 |
| | | Carbopol 974P | 5 |

¹Percent by weight based upon the weight of the solution.

Anticaries and bleach agents were added to the indicated liquid and mixed to form a clear solution. The following Table 3 shows the composition of each of these starting solutions.

TABLE 3
Starting Solutions 1AC, 2AC, and 1B

| Starting | | | Agent Concentration |
|---|---|---|---|
| Solution | Liquid | Agent | Weight Percent¹ |
| 1AC | Glycerol | xylitol | 5.0 |
| 2AC | Glycerol | xylitol | 10 |
| 1B | Glycerol | UHP | 15 |

¹Percent by weight based upon the weight of the solution.

### Preparation of Mixed Preparative Compositions

Selected starting solutions described above were combined with another of these starting solutions and/or with another material with mixing to provide mixed preparative compositions. All mixed preparative compositions were clear, except for mixed preparative composition 2MXPC, which was turbid. The following Tables 4 and 4A show the composition of each of these mixed preparative compositions. These compositions were aged under ambient conditions and observed periodically, the results of which are shown in Table 5. Compositions 2MXPC and 3MXPC were tested for release of calcium and fluoride ions according to the Ion Release test method described above. Results are shown in Table 15 below. The composition 3MXPC was tested for its ability to recharge a coating with calcium ions according to the Ion Recharge Of Coatings test method described above. Results are shown in Table 17 below. The composition 3MXPC was also tested for its ability to occlude dentin tubules according to the Dentin Tubule Occlusion test method described above. A slurry made from the composition and water in a ratio of 1:1 was gently swabbed on the exposed dentin slab with a premoistened cotton swab for 2 minutes. SEM's of the treated dentin showed tubule occlusion.

TABLE 4
Mixed Preparative Compositions (MXPC) 1MXPC-4MXPC

| MXPC | 1C | 3C | 4C | 1P | 10P | 11P | 15P | 2MFC |
|---|---|---|---|---|---|---|---|---|
| 1MXPC | 3.2 g | | | | | | | 9.2 g |
| 2MXPC | | 12.6 g | | | | | 17.4 g | |
| 3MXPC | | 14.2 g | | | 15.2 g | | | |
| 4MXPC | | | 1.8 g | | | 1.8 g | | |

TABLE 4A
Mixed Preparative Compositions (MXPC) 5MXPC-8MXPC

| MXPC | 1C | 1P | 3MFC | 1B | Ca(NO₃)₂•4H₂O | Glycerol | SGP¹ |
|---|---|---|---|---|---|---|---|
| 5MXPC | | | 16.5 g | | 4.5 g | | |
| 6MXPC | 8.1 g | 2.3 g | | | | 7 g | |
| 7MXPC | | | | 10 pbw² | 0.173 pbw | | |
| 8MXPC | | | | 10 pbw | | | 0.142 pbw |

¹SGP = (NaO)₂(O)POCH(CH₂OH)₂•xH₂O
²pbw = parts by weight

TABLE 5
Observations After Aging Mixed Preparative Compositions
1MXPC-8MXPC

| Mixed Starting Composition | Observation |
|---|---|
| 1MXPC | Clear after 26 months of aging |
| 2MXPC | Separated after 4 months |
| | of aging |
| 3MXPC | Clear after 4 months of aging |
| 4MXPC | Clear after 13 months of aging |
| 5MXPC | Clear after 25 months of aging |
| 6MXPC | Clear after 13 months of aging |
| 7MXPC | Clear after 2 months of aging |
| 8 MXPC | Clear after 2 months of aging |

### Polymerizable Preparative Compositions

Metal ion-containing polymerizable compositions were prepared by combining 98.25% of an 80:20 blend of UVR-6105 and pTHF (mixed at 600 rpm for 10 minutes on ice using a Vertishear Cyclone I.Q.) with 0.5% CPQ and 1.25% DPISbF₆ and mixing at 15,000 rpm for 40 minutes on ice using a Vertishear Cyclone I.Q. To the resulting polymerizable resin was added 10 weight percent of a metal methacrylate to provide the polymerizable preparative compositions shown in Table 6. The fluorescent behavior of cured disks of each composition was observed under illumination by a Spectroline ENF-260C long wavelength UV light (Spectronics Corp., Westbury, N.Y.). Rheological properties and fluorescence of these compositions are shown in Table 6. The rare earth and zinc additives can be used, for example, in compositions where radiopacity is desired. The europium additive can be used, for example, in compositions where visibly distinguishing the composition from the tooth structure is desired.

TABLE 6
Metal Ion-Containing Preparative Polymerizable Compositions (MIPPC),
Rheological Properties, and Fluorescence

| | Metal | | |
|---|---|---|---|
| | Methacrylate (10 | Rheological | |
| MIPPC | Weight Percent) | Properties | Fluorescence |
| 1MIPPC | Yttrium | Gelatinous | Bright |
| | methacrylate* | | yellow |
| 2MIPPC | Europium | Gelatinous | Pale red |
| | methacrylate* | fluid | |
| 3MIPPC | Zirconium | Soft wax- | Bright |
| | methacrylate* | like | yellow |
| 4MIPPC | Zinc | Hard wax- | Bright |
| | methacrylate** | like | yellow |

*Available from Gelest Inc., Morrisville, PA
**Available from Rohm-Tech, Malden, MA

Each of the above starting solutions and preparative compositions can be used as a part of a composition described herein, either by combining two or more of these starting solutions and/or compositions, or by providing two or more of these starting solutions and/or compositions as parts of a multi-part composition, for example, a 2-part composition.

### Examples 1-8

The indicated starting solutions and other materials were combined in the amounts shown in Table 7 and mixed to form compositions, which were clear unless otherwise indicated. The compositions were aged under ambient conditions and observed periodically.

TABLE 7
Compositions and Observations After Aging of Examples 1-7

| Example | 1C | 1P | 1MFC | 3MFC | IADMA | Observation |
|---|---|---|---|---|---|---|
| 1 | 55.7 g | 14.9 | 7.1 | — | — | Clear at 100 |
| | | | | | | days |
| 2 | 55.9 g | 14.8 g | — | — | 7.1 g | Clear at 100 |
| | | | | | | days |
| 3 | 0.15 g | 0.07 g | — | — | 2.2 g | Clear at 100 |
| | | | | | | days |
| 4 | 55.7 g | 14.9 g | — | — | 7.1 g | Clear at 100 |
| | | | | | | days |
| 5 | 0.15 g | 0.07 g | 2.2 g | — | — | Clear at 60 |
| | | | | | | days |
| 6 | 0.74 g | 0.44 g | 11.1 g | — | — | Clear at 13 |
| | | | | | | days, ppts¹ |
| | | | | | | at 73 days |
| 7 | 6.3 g | 2.7 g | — | 9.0 g | — | Ppts at 13 |
| | | | | | | days |
| 8 | 36 g | 9.65 g | 4.56 g | — | — | — |

¹Some precipitate was evident.

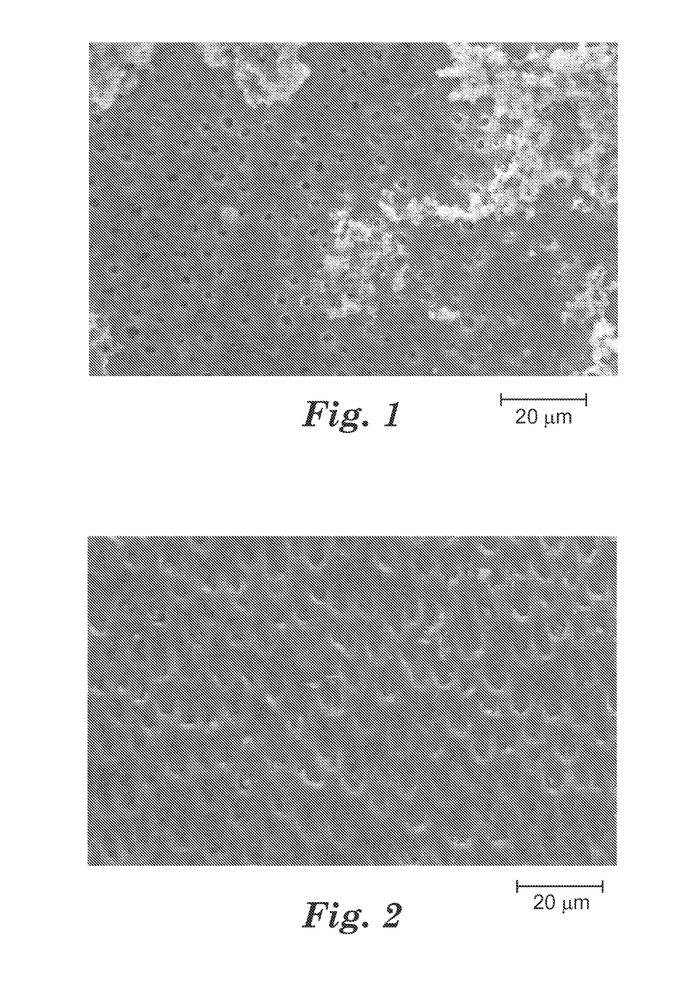

The composition of Example 8 was used to treat exposed dentin according to the Dentin Tubule Occlusion test method described above. The composition was applied with a fibertip. Partial occlusion of dentin tubules after one treatment was found as shown in FIG. 1. The occlusion of the tubules appeared to be the result of surface deposition.

### Example 9

A blend of starting solutions 1C (320 g) and 1P (81.7 g) was prepared by combining and mixing the solutions. A portion (0.4 g) of the resulting clear solution was mixed with starting solution 4MFC (8.5 g) to provide a slightly turbid solution. After aging at ambient conditions for 33 months, the solution was slightly turbid and appeared to be unchanged.

### Example 10

A blend of starting solutions 1C (3.3 g) and 2P (33.7 g) was prepared by combining and mixing the solutions. A portion (1.2 g) of the resulting clear solution was mixed with starting solution 3MFC (1.2 g) to provide a clear solution. After aging at ambient conditions for 11.5 months, the solution was still clear.

### Example 11

A blend of starting solutions 13C (1.9 g) and 23P (2.0 g) was prepared by combining and mixing the solutions to provide a clear composition. After aging at ambient conditions for 24 months, the clear composition was still clear.

### Example 12

Calcium nitrate (4 parts by weight (pbw)) was fully dissolved in CPVH resin (92 pbw) with mixing. Sodium hexafluorophosphate (4 pbw) was mixed with to the calcium nitrate solution in CPVH resin to provide a clear composition. After aging at ambient conditions for 24 months, the clear composition was still clear.

### Example 13

Calcium nitrate (5 pbw) and sodium hexafluorophosphate (5 pbw) were combined with 3CPVH resin (90 pbw) with mixing to provide a clear composition. After aging at ambient conditions for 24 months, precipitate had formed in the composition.

### Example 14

Starting solution 15C (1.8 g) was combined with starting solution 24P (1.5 g) with mixing to provide a clear composition. After aging at ambient conditions for 24 months, precipitate had formed in the composition.

### Examples 15-24

The indicated starting solutions and the Example 15 composition were combined in the amounts shown in Table 8 and mixed to provide compositions, which were clear. The solutions were aged under ambient conditions and observed periodically, and the results are recorded in Table 9.

TABLE 8
Compositions of Examples 15-24

| Ex¹ | 3C | 4C | 6C | 3P | 4P | 3MI | 4MI | 1B | Ex 15 |
|---|---|---|---|---|---|---|---|---|---|
| 15 | | 2.4 g | | | 6 g | | | | |
| 16 | | 1.1 g | | | 1.9 g | | | | |
| 17 | | 0.4 g | | | 0.4 g | | | 1.3 g | |
| 18 | | 3.7 g | | | 1.7 g | | | | |
| 19 | | 2 g | | | 2.1 g | 0.4 g | | | |
| 20 | | | | | | | 0.4 g | | 0.9 g |
| 21 | | | 1.5 g | | 0.9 g | | | | |
| 22 | 13.5 g | | | 14.6 g | | 1.9 g | | | |
| 23 | 7.1 g | | | 9.7 g | | 1.9 g | 11.2 g | | |
| 24 | 24.1 g | | | 38.3 g | | 6.3 g | 37.3 g | | |

¹Ex = Example

TABLE 9
Observations After Aging Compositions of Example 15-24.

| Example | Observation |
|---|---|
| 15 | Precipitate observed after 5 months of aging |
| 16 | Clear after 5 months of aging |
| 17 | Clear after 5 months of aging |
| 18 | Clear after 5 months of aging |
| 19 | Clear after 5 months of aging |
| 120 | Clear after 5 months of aging |
| 21 | Clear after 3 months of aging |
| 22 | Turbidity observed after 4 months of aging |
| 23 | Clear after 4 months of aging |
| 24 | Not observed after aging |

The compositions of Examples 22 and 23 were tested for release of calcium and fluoride ions according to the Ion Release test method described above. Results are shown in Table 15 below.

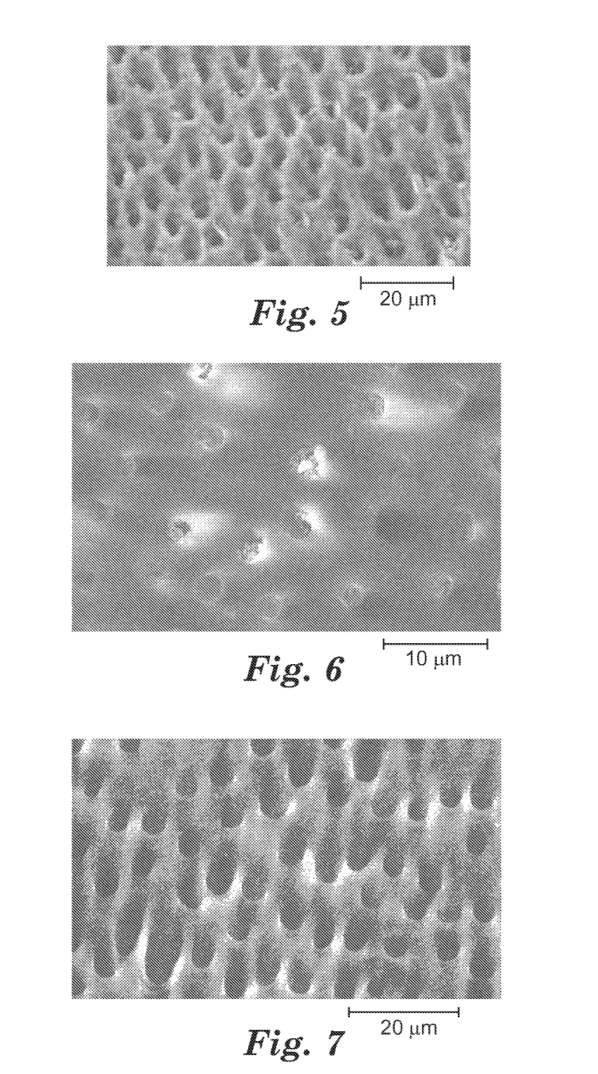

The composition of Examples 22 and 23 were used to treat exposed dentin according the Dentin Tubule Occlusion test method described above. A slurry made from the composition and water in a ratio of 1:1 was gently swabbed on the exposed dentin slab with a premoistened cotton swab. Partial occlusion of dentin tubules after one treatment was found as shown in FIGS. 2 and 5, respectively. The occlusion of the tubules appeared to be the result of precipitation of particles within the tubules for Example 23 and both precipitation of particles within the tubules and surface deposition for Example 22.

### Examples 25-31

The indicated starting solutions were combined in the amounts shown in Table 10 and mixed to form compositions, which were clear. The solutions were aged under ambient conditions and observed periodically, and the results are recorded in Table 11.

TABLE 10
Compositions of Examples 25-31

| Example | 4C | 5C | 7C | 8C | 4P | 5P | 6P | 7P | 8P | 9P |
|---|---|---|---|---|---|---|---|---|---|---|
| 25 | | | 2.0 g | | 1.1 g | 1.7 g | | | | |
| 26 | | | 3.9 g | | | 3.1 g | | | | |
| 27 | 1.1 g | | | | 1.1 g | | 0.4 g | | | |
| 28 | 2.6 g | | | | | | | 2.3 g | | |
| 29 | | | | 1.2 g | | | | | 0.9 g | |
| 30 | | 4.1 g | | | | | | | 2.2 g | |
| 31 | 0.7 g | | | | 1.4 g | | | | | 0.4 g |

TABLE 11
Observations After Aging Compositions of Example 25-31

| Example | Observation |
|---|---|
| 25 | Clear after 3 months of aging |
| 26 | Clear after 3 months of aging |
| 27 | Clear after 5 months of aging |
| 28 | Clear after 13 months of aging |
| 29 | Clear after 13 months of aging |
| 30 | Clear after 13 months of aging |
| 31 | Clear after 13 months of aging |

### Examples 32-37

The indicated starting solutions and the Example 15 composition were combined in the amounts shown in Table 12 and mixed to form compositions, which were clear except for Example 37 which was slightly turbid. The solutions were aged under ambient conditions and observed periodically, and the results are recorded in Table 13.

TABLE 12
Compositions of Examples 32-37

| | | | | | | | | | | | Example |
|---|---|---|---|---|---|---|---|---|---|---|---|
| Example | 4C | 10C | 11C | 4P | 9P | 11P | 19P | 21P | 3MI | 4MI | 15 |
| 32 | 1.0 g | | | | 0.3 g | 0.7 g | | | | | |
| 33 | | | | | 0.4 g | | | | | | 1.1 g |
| 34 | 1.1 g | | | | 1.1 g | | | | 0.3 g | 0.5 g | |
| 35 | | 1.6 g | | | 0.7 g | | | | | | |
| 36 | | | 2.0 g | | | | 0.4 g | | | | |
| 37 | | 2.3 g | | 1.1 g | | | | 0.4 g | | | |

TABLE 13
Observations After Aging Compositions of Example 32-37.

| Example | Observation |
|---|---|
| 32 | Clear after 5 months of aging |
| 33 | Clear after 5 months of aging |
| 34 | Clear after 3 months of aging |
| 35 | Clear after 5 months of aging |
| 36 | Clear after 5 months of aging |
| 37 | Slightly turbid after 5 months of aging |

### Examples 38-43

The indicated starting solutions and compositions of Examples 8 and 24 were combined in the amounts shown in Table 14 and mixed to form compositions, which were clear.

TABLE 14
Compositions of Examples 36-43

| Exam- | | | | | | | Exam- | Example |
|---|---|---|---|---|---|---|---|---|
| ple | 2MI | 12MI | 13MI | 14MI | 15MI | 16MI | ple 8 | 24 |
| 38 | 0.5 g | | | | | | | 3.7 g |
| 39 | | 0.9 g | | | | | 2.5 g | |
| 40 | | | 0.8 g | | | | 2.5 g | |
| 41 | | | | 0.8 g | | | 2.5 g | |
| 42 | | | | | 0.9 g | | 2.9 g | |
| 43 | | | | | | 1.0 g | 2.5 g | |

### Example 44

Starting solution 4C (0.9 g) was combined with starting solutions 4P (0.9 g), 6P (0.3 g), and 1B (1.8 g) with mixing to provide a clear composition. After aging at ambient conditions for 5 months, the clear composition was still clear.

### Example 45

Sodium glycerophosphate ((NaO)₂(O)POCH(CH₂OH)₂.xH₂O) (0.0646 g) was combined with starting solution 1F (6.4 g) with mixing to provide a clear composition (1% sodium glycerophosphate, 0.4% SnF₂, glycerol).

### Example 46

Starting solution 3C (2.3 g) was combined with starting solution 2F (2.7 g) with mixing to provide a clear composition (4.6% Ca(NO₃)₂.4H₂O, 0.4% SnF₂, glycerol).

### Example 47

Starting solution 3C (1.3 g) was combined with starting solutions 16P (2.1 g) and 4F (7.8 g), and with glycerol (9.1 g) with mixing to provide a clear composition (0.64% Ca(NO₃)₂.4H₂O, 0.21% NaH₂PO₂.H₂O, 0.63% SnF₂, glycerol).

### Example 48

Starting solution 3C (1.32 g) was combined with starting solutions 31P (1.0 g) and 4F (7.8 g), and with glycerol (9.9 g) with mixing to provide a clear composition (0.65% Ca(NO₃)₂.4H₂O, 0.20% H₄P₂O₇, 0.64% SnF₂, glycerol).

### Example 49

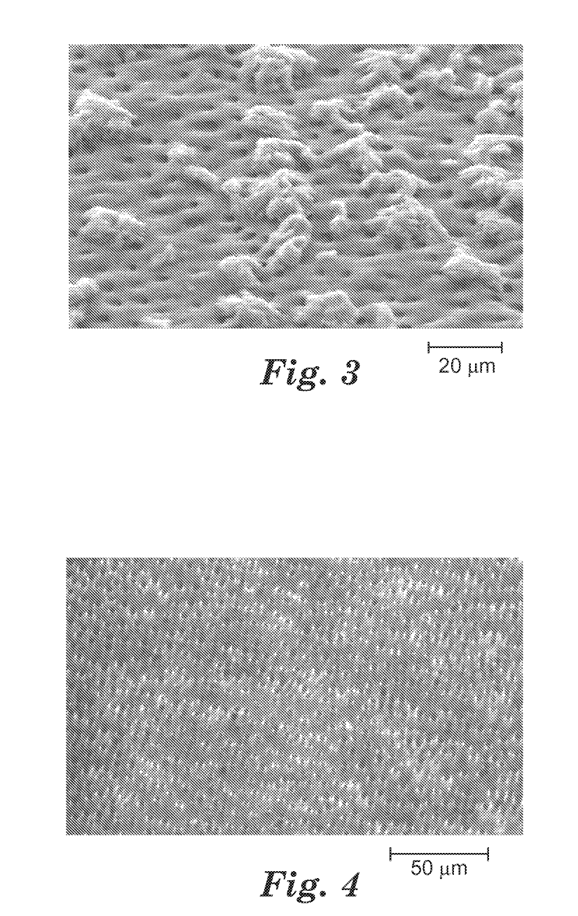

Starting solution 3C (8.3 g) was combined with starting solution 16P (12.2 g) with mixing to provide a clear composition (4.0% Ca(NO₃)₂.4H₂O, 1.19% NaH₂PO₂.H₂O, glycerol). The composition was used to treat exposed dentin according to the Dentin Tubule Occlusion test method described above. A slurry made from the composition and water in a ratio of 1:1 was gently swabbed on the exposed dentin slab with a premoistened cotton swab. Partial occlusion of dentin tubules after one treatment was found as shown in FIG. 3. The occlusion of the tubules appeared to be the result of surface deposition.

### Example 50

Starting solution 3C (8.9 g) was combined with starting solution 31P (10.3 g) and with glycerol (1.5 g) with mixing to provide a clear composition (4.3% Ca(NO₃)₂.4H₂O, 2.0% H₄P₂O₇, glycerol).

### Example 51

Starting solution 3C (1.18 pbw) was combined with starting solutions 3P (1.30 pbw), and 1F (7.84 pbw)) with mixing to provide a clear composition (0.11%

Ca(NO₃)₂.4H₂O, 0.13% (NaO)₂(O)POCH(CH₂OH)₂.xH₂O, 0.30% SnF₂, glycerol). This composition was tested for release of calcium and fluoride ions according to the Ion Release test method described above. Results are shown in Table 15 below.

### Example 52

Anhydrous dicalcium phosphate (CaHPO₄) was combined with the composition of Example 22 with mixing to provide a paste composition containing 50% (weight percent) dicalcium phosphate (2.25% Ca(NO₃)₂.4H₂O, 0.15% Zn(NO₃)₂.6H₂O, 2.43% (NaO)₂(O)POCH(CH₂OH)₂.xH₂O, 50% CaHPO₄, glycerol). This composition was tested for release of calcium and fluoride ions according to the Ion Release test method described above. Results are shown in Table 15 below.

### Comparative Example 1

A sufficient amount of anhydrous dicalcium phosphate was combined with glycerol with mixing to provide a paste composition containing 50% (weight percent) dicalcium phosphate. This composition was tested for release of calcium and fluoride ions according to the Ion Release test method described above. Results are shown in Table 15 below.

This preparative composition can be used as a part of a composition described herein, either by combining this composition with one or more starting solutions, preparative compositions, or Example compositions described herein, or by providing this composition as a part of a multi-part composition, for example, a 2-part composition.

### Comparative Example 2

CREST Rejuvenating Effects Fluoride Anticavity Toothpaste, energizing mint flavor (Procter & Gamble, Cincinnati, Ohio) was tested for release of calcium and fluoride ions according to the Ion Release test method described above. Results are shown in Table 15 below.

### Comparative Example 3

SOOTHERX paste was tested for release of calcium and fluoride ions according to the Ion Release test method described above. Results are shown in Table 15 below.

### Comparative Example 4

PROSPEC MI paste was tested for release of calcium and fluoride ions according to the Ion Release test method described above. Results are shown in Table 15 below.

TABLE 15
Calcium And Fluoride Ion Release From Composition Of Examples 22,
23, 51, 52 and Comparative Example 1

Micrograms Ion Released/
Gram of Paste or Gel
at 1 Minute

| Example | Calcium Ion | Fluoride Ion |
|---|---|---|
| 22 | 463.8 | 0.6 |
| 23 | 993.0 | 0.2 |
| 51 | 81.6 | 237.8 |
| 52 | 1365.0 | 0.2 |
| Comparative Example 1 | 204.3 | 0.4 |
| Comparative Example 2 | 0.4 | 706.5 |
| Comparative Example 3 | 0.6 | 2.0 |
| Comparative Example 4 | 18.9 | 0.5 |

TABLE 16
Calcium And Fluoride Ion Release From Mixed Preparative
Compositions 2MXPC and 3MXPC

| | |
|---|---|
| Mixed | Micrograms Ion Released/ |
| Preparative | Gram of Paste or Gel at 1 Minute |

| Composition | Calcium Ion | Fluoride Ion |
|---|---|---|
| 2MXPC | 343.5 | 0.2 |
| 3MXPC | 2664.0 | 0.4 |

All of the Example compositions and preparative compositions tested showed higher levels of calcium ion release than the calcium-containing commercial products represented by Comparative Examples 3 and 4. Example 52, which includes both calcium and phosphorous releasing particles and solubilized calcium and phosphate, showed much higher calcium release than Comparative Example 1, which included only the particulate source, and Example 22, which included only the solubilized salts.

### Example 53

The composition of Example 6 was coated onto the frosted surface of a glass slide using a fiber-tipped brush (available from 3M ESPE), and the coating was allowed to air-dry for 30 minutes. The resulting coating was tested for calcium ion release as described in the “Ion Release From Coatings” test method described above. The results are shown in Table 17.

### Example 54

The composition of Example 7 was made into a coating on a glass slide and tested for calcium ion release as in Example 53. The results are shown in Table 17.

### Comparative Example 5

IADMA was made into a coating on a glass slide and tested for calcium ion release as in Example 53. The results are shown in Table 17.

### Comparative Example 6

Preparative solution 3MFC was made into a coating on a glass slide and tested for calcium ion release as in Example 53. The results are shown in Table 17.

TABLE 17
Ion Release From Coatings Of Examples 53 and 54 and
Comparative Examples 5 and 6.

| | Cumulative Micrograms Calcium | | |
|---|---|---|---|
| | Ion Released/Gram Composition | | |
| Example | 0.33 Hours | 2 Hours | 24 Hours |
| 53 | 11820 | 11820 | 18170 |
| 54 | 92810 | 101483 | 106047 |
| Comparative | 6914 | 6914 | 12333 |
| Example 5 | | | |
| Comparative | 2920 | 4762 | 6682 |
| Example 6 | | | |

### Example 55

A slurry of the Example 22 gel composition and water (1:1) was swabbed on the surface of a coating of IADMA on a glass slide, and calcium ion recharge of the coating were carried out according to the Ion Recharge Of Coatings test method described above. Results are shown in Table 18 below.

### Example 56

A slurry of the Example 41 gel composition and water (1:1) was swabbed on the surface of a coating of IADMA on a glass slide, and calcium ion recharge of the coating were carried out according to the Ion Recharge Of Coatings test method described above. Results are shown in Table 18 below.

### Example 57

A gel composition was prepared by combining Starting Solutions 3C (4.15 g) and 15P (8.68 g) with 0.5% SnF₂ in glycerol (37.24 g). A slurry of the resulting gel composition and water (1:1) was swabbed on the surface of a coating of IADMA on a glass slide, and calcium ion recharge of the coating was carried out according to the “Ion Recharge Of Coatings” test method described above. Results are shown in Table 18 below.

### Comparative Example 7

A slurry of SOOTHERX paste and water (1:1) was swabbed on the surface of a coating of IADMA on a glass slide, and calcium ion recharge of the coating were carried out according to the Ion Recharge Of Coatings test method described above. Results are shown in Table 18 below.

### Comparative Example 8

A slurry of PROSPEC MI paste and water (1:1) was swabbed on the surface of a coating of IADMA on a glass slide, and calcium ion recharge of the coating were carried out according to the Ion Recharge Of Coatings test method described above. Results are shown in Table 18 below.

TABLE 18
Calcium Ion Recharge Of IADMA Coating Using Compositions Of
Examples 22, 51, and the gel composition of Example 57, Mixed
Preparative Composition 3MXPC, SOOTHERX paste, and
PROSPEC MI paste.

| Example or | | Calcium (ppm) | | |
|---|---|---|---|---|
| Comparative | Composition Used For | | Time = | Time = |
| Example | Recharge | Time = 0 | 24 hr | 48 hr |
| 55 | Example 22 | 0 | 1700 | 8300 |
| 56 | Example 51 | 0 | 0 | 5200 |
| 57 | Gel Composition In | 0 | 0 | 7600 |
| | Example 57 | | | |
| — | 3MXPC | 0 | 0 | 6000 |
| Comp. Ex. 7 | SOOTHERX paste | 0 | 0 | 4500 |
| Comp. Ex. 8 | PROSPEC MI paste | 0 | 0 | 3500 |

### Example 58

The composition of Example 24 was used as the first part of a 2-part composition. The second part was POSTPROPHY Treatment Solution A. A 1:1 (wt/wt) blend of the first part and the second part was prepared. The resulting mixture was used to treat exposed dentin according to the Dentin Tubule Occlusion test method described above. The mixture was gently swabbed on the exposed dentin slab with a premoistened cotton swab. Partial occlusion of dentin tubules after one treatment was found as shown in FIG. 6. The occlusion of the tubules appeared to be the result of precipitation of particles within the tubules.

Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

1. A remineralizing composition comprising:
a calcium source and a phosphorous source, each of which is dissolved in a substantially anhydrous liquid; and
a matrix forming component selected from the group consisting of a polymerizable resin, a film former, and a combination thereof;
wherein the matrix forming component is dissolved in the substantially anhydrous liquid, and wherein the matrix forming component optionally comprises a portion of the substantially anhydrous liquid; or
wherein the matrix forming component is the substantially anhydrous liquid.

2. The composition of claim 1, further comprising at least one metal cation selected from the group consisting of cations of Mg, Sr, Ba, Sn, Zn, Zr, La, Al, and Ag.

3. The composition of claim 1 wherein the composition is a one-part composition.

4. The composition of claim 1 wherein the composition is a two-part composition, and wherein the calcium source is in one part and the phosphorous source is in the other part.

5. The composition of claim 1, wherein the matrix forming component is a polymerizable resin.

6. The composition of claim 1, wherein the matrix forming component is a film former.

7. The composition of claim 1 wherein the film former is a polyacid selected from the group consisting of a homopolymer of a monomer, a copolymer of two or more different monomers, and a combination thereof, wherein the monomer and the two or more different monomers are selected from the group consisting of acrylic acid, methacrylic acid, itaconic acid, maleic acid, glutaconic acid, aconitic acid, citraconic acid, mesaconic acid, fumaric acid, and tiglic acid.

8. The composition of claim 7, wherein the polyacid further comprises a pendent polymerizable group.

9. A remineralizing composition comprising:
a calcium source, a phosphorous source, and at least one cation selected from the group consisting of cations of Zn, Sn, and Ag, wherein the calcium source, the phosphorous source, and the at least one cation are dissolved in a substantially anhydrous liquid.

10. The composition of claim 9, further comprising at least one cation selected from the group consisting of cations of Mg, Ba, and Sr.

11. The composition of claim 9, further comprising a second part, wherein the second part is an orally acceptable liquid or paste.

12. The composition of claim 1, further comprising an anticaries agent.

13. A remineralizing composition comprising at least one divalent metal cation and a phosphate anion, both of which are dissolved in a substantially anhydrous liquid;
wherein the phosphate anion is selected from the group consisting of (P₂O₇)⁻⁴, (H₂PO₂)⁻¹, and an anion represented by the formula: H—[CH(—OR)—]ₓH, wherein x is an integer from 2 to 4; and each R is independently H or —P(O)(O⁻)₂, and wherein at least one R is —P(O)(O⁻)₂.

14. The composition of claim 13, wherein the phosphate anion together with the at least one divalent metal cation is a salt which has a lower solubility in the substantially anhydrous liquid than that of either the divalent metal cation or the phosphate anion on a molar basis at 25° C.

15. The composition of claim 14, wherein the phosphate anion together with the at least one divalent metal cation is a salt which is insoluble in the substantially anhydrous liquid when the salt is combined with the substantially anhydrous liquid.

16. The composition of claim 13, wherein the phosphate anion is a glycerophosphate.

17. The composition of claim 13, wherein the divalent metal cation is selected from the group consisting of divalent cations of Ca, Zn, Sn, Sr, Mg, and Ba.

18. A method of preparing a remineralizing composition comprising:
dissolving a phosphate anion in a substantially anhydrous liquid to provide a first solution; wherein the phosphate anion is selected from the group consisting of (P₂O₇)⁻⁴, (H₂PO₂)⁻¹, and an anion represented by the formula: H—[CH(—OR)—]ₓH, wherein x is an integer from 2 to 4; and each R is independently H or —P(O)(O⁻)₂, and wherein at least one R is —P(O)(O⁻)₂;
dissolving at least one divalent metal cation separately from the phosphate anion in the substantially anhydrous liquid to form a second solution; and
combining the first and second solutions.

19. The method of claim 18, wherein the divalent metal cation is selected from the group consisting of Ca, Zn, Sn, Sr, Mg, and Ba.

20. A composition comprising a divalent metal cation source and an organic anticaries agent, both of which are dissolved in a substantially anhydrous liquid, wherein the divalent metal cation is selected from the group consisting of divalent cations of Ca, Zn, Sr, Ba, and Mg.

21. The composition of claim 20, further comprising a phosphorous source dissolved in the substantially anhydrous liquid.

22.-65. (canceled)
